(19) United States
(12) Patent Application Publication
(10) Pub. No.: US 2025/0092051 A1
(43) Pub. Date: Mar. 20, 2025

(54) NLRP3 MODULATORS AND USE THEREOF

(71) Applicants: Chunqiang Ye, Beijing (CN); Wei Wang, Belmont, MA (US); Youdong Mao, Malden, MA (US)

(72) Inventors: Chunqiang Ye, Beijing (CN); Wei Wang, Belmont, MA (US); Youdong Mao, Malden, MA (US)

(73) Assignee: Bioentropy Inc., Cambridge, MA (US)

(21) Appl. No.: 18/830,366

(22) Filed: Sep. 10, 2024

Related U.S. Application Data
(60) Provisional application No. 63/537,804, filed on Sep. 11, 2023.

(51) Int. Cl. C07D 487/04; A61K 31/519; A61K 31/5377

(52) U.S. Cl. CPC C07D 487/04; A61K 31/519; A61K 31/5377

(57) ABSTRACT

The present invention relates to novel compounds that modulate the NLRP3 inflammasome pathway, specifically targeting the NOD-like receptor protein 3 (NLRP3). These compounds, including 8-azahypoxanthine derivatives and their pharmaceutically acceptable salts, hydrates, and drug combinations, demonstrate efficacy in inhibiting NLRP3 activation. Such modulation has potential therapeutic applications for a variety of diseases associated with NLRP3-mediated inflammation, including cancer, inflammatory, cardiovascular, and neurodegenerative conditions. The invention also includes methods for synthesizing these compounds, pharmaceutical compositions containing them, and their potential use for treating diseases like Alzheimer's, Parkinson's, multiple sclerosis, and asthma.

### EXAMPLES

### Example 1: Preparation of 2-(4-bromo-2,6-dimethylphenyl)-5-morpholino-2,6-dihydro-7H-[1,2,3]triazolo[4,5-d]pyrimidin-7-one (Compound 1)

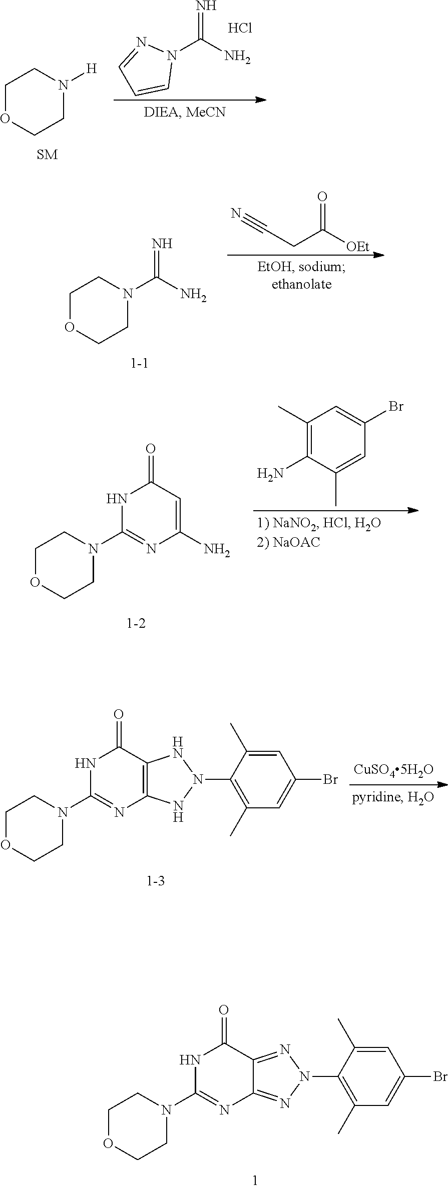

### Compound 1-1: morpholine-4-carboximidamide

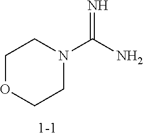

To a solution of morpholine (2.00 g, 22.9 mmol, 2.02 mL) and pyrazole-1-carboxamidine (3.36 g, 22.9 mmol) in ACN (50 mL) was added DIEA (2.97 g, 22.9 mmol, 4.00 mL). The mixture was stirred at 20° C. for 12 hrs. TLC (CH₂Cl₂/MeOH=8/1, Rf=0.2) showed the reaction was completed. The reaction mixture was filtered and the solid was drying to yield morpholine-4-carboximidamide (3 g, crude) as a white solid.

1H NMR: (400 MHz, DMSO-d6) δ 3.71-3.77 (m, 4H), 3.44-3.50 (in, 4H).

### Compound 1-2: 6-amino-2-morpholinopyrimidin-4(3H)-one

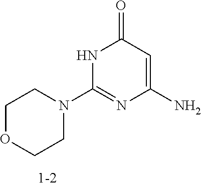 [SMILES: Nc1cc(=O)[nH]c(N2CCOCC2)n1]

To a solution of morpholine-4-carboxamidine (2 g, 6.04 mmol) and ethyl 2-cyanoacetate (1.02 g, 9.06 mmol, 965 L) in EtOH (20 mL) was added sodium; ethanolate (4.11 g, 12.0 mmol, 20% purity). The mixture was stirred at 85° C. for 4 hrs. TLC (CH₂Cl₂/MeOH=10/1, Rf=0.3) showed the reaction was completed. The reaction mixture was filtered, and the filtrate was concentrated to yield a residue, which was purified by column chromatography (SiO₂, CH₂Cl₂: MeOH=100/1 to 10/1) to give 6-amino-2-morpholinopyrimidin-4(3H)-one (1.4 g, 5.35 mmol) as a white solid.

1H NMR: (400 MHz, DMSO-d6) δ 9.30-10.71 (m, 1H), 7.78-7.94 (m, 1H), 6.04 (s, 2H), 3.58 (d, J=4.4 Hz, 4H), 3.51 (d, J=4.8 Hz, 4H).

### Compound 1-3: 2-(4-bromo-2,6-dimethylphenyl)-5-morpholino-1,2,3,6-tetrahydro-7H-[1,2,3]triazolo[4,5-d]pyrimidin-7-one

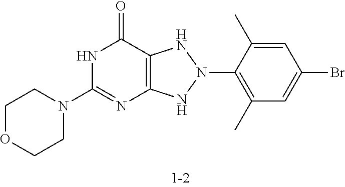 [SMILES: Cc1cc(Br)cc(C)c1N1Nc2nc(N3CCOCC3)[nH]c(=O)c2N1]

To a solution of 4-bromo-2,6-dimethyl-aniline (200 mg, 999 mol) in Water (10 mL) and HCl (5 mL) was added A solution of NaNO₂ (68.9 mg, 999 mol) of sodium nitrite in 2 ml of water is added dropwise over a period of 2 minutes. The reaction solution is stirred at 0-5° C. for another 30 minutes and is subsequently added to a suspension of 6-amino-2-morpholinopyrimidin-4(3H)-one (196 mg, 999 mol) and NaOAc (82.0 mg, 999 μmol) of sodium acetate in 3 ml of water. The solution is stirred for 2 hrs at 0-5° C. The mixture was stirred at 25° C. for 12 hrs. LC-MS showed the reaction was completed. The reaction mixture was the filtrate was concentrated to yield 2-(4-bromo-2,6-dimethylphenyl)-5-morpholino-1,2,3,6-tetrahydro-7H-[1,2,3]triazolo[4,5-d]pyrimidin-7-one (230 mg, 376 mol, 37.6% yield) as a yellow solid.

LC-MS: m/z=409.0 (M+H)⁺, Rt=1.143 min.

### Compound 1: 2-(4-bromo-2,6-dimethylphenyl)-5-morpholino-2,6-dihydro-7H-[1,2,3]triazolo[4,5-d]pyrimidin-7-one

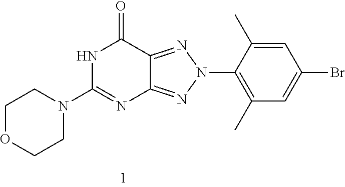 [SMILES: Cc1cc(Br)cc(C)c1-n1nc2nc(N3CCOCC3)[nH]c(=O)c2n1]

To a solution of 2-(4-bromo-2,6-dimethyl-phenyl)-5-morpholino-3,3a,6,7a-tetrahydro-1H-triazolo[4,5-d]pyrimidin-7-one (250 mg, 610 mol) in PYRIDINE (7 mL). The temperature is subsequently increased to 75° C., was added dropwise CuSO₄·5H₂O (686 mg, 2.75 mmol) in 7 ml of water which is at a temperature of 70° C. The resulting mixture was stirred at 100° C. for 12 hrs. LC-MS showed the reaction was completed. The reaction mixture was diluted with water (20 mL) and extracted with EtOAc (20 mL×3). The combined organic layers were dried over Na₂SO₄, filtered and the filtrate was concentrated to yield a residue, which was purified by prep-HPLC (column: Xtimate C18 150*40 mm*10 μm; mobile phase: [water(HCl)-ACN]; gradient: 24%-64% B over 36 min) to give 2-(4-bromo-2,6-dimethylphenyl)-5-morpholino-2,6-dihydro-7H-[1,2,3]triazolo-[4,5-d]pyrimidin-7-one (13.0 mg, 48.9 mol, 5.51% yield, 99.1% purity) as a white solid.

¹H NMR: (400 MHz, DMSO-d6) δ 11.51 (s, 1H), 7.58 (s, 2H), 3.64-3.71 (m, 4H), 3.58-3.64 (m, 4H), 1.96 (s, 6H).

LC-MS: m/z=414.5 (M+H)⁺, Rt=2.754 min.

### Example 2: Preparation of 2-(4-bromo-2,6-dimethylphenyl)-5-morpholino-2,6-dihydro-7H-[1,2,3]triazolo[4,5-d]pyrimidine-7-thione (Compound 2)

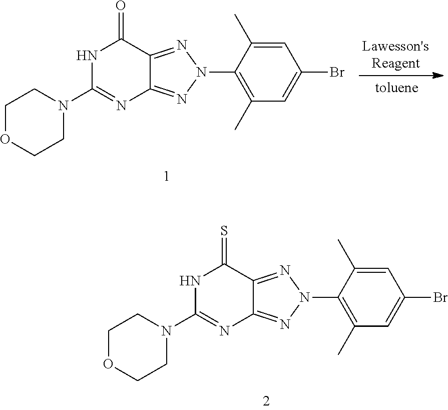 [SMILES: Cc1cc(Br)cc(C)c1-n1nc2nc(N3CCOCC3)[nH]c(=O)c2n1 ; Cc1cc(Br)cc(C)c1-n1nc2nc(N3CCOCC3)[nH]c(=S)c2n1]

To a green solution of 2-(4-bromo-2,6-dimethyl-phenyl)-5-morpholino-6H-triazolo [4,5-d]pyrimidin-7-one (500 mg, 1.23 mmol) in toluene (10 mL) was added 2,4-bis(4-methoxyphenyl)-2,4-dithioxo-1,3,2,4dithiadiphosphetane (1.50 g, 3.70 mmol). The mixture was stirred at 120° C. for 2 hrs. LC-MS showed Reactant 1 was consumed completely. TLC (CH₂Cl₂/MeOH=10:1, UV) indicated Reactant 1 was consumed completed. The reaction mixture was filtered and concentrated under reduced pressure to give a residue, which was purified by prep-HPLC (HCl condition: column: Xtimate C18 150*40 mm*10 μm; mobile phase: [water(HCl)-MeOH]; gradient: 48%-88% Bover 36 min), to give 2-(4-bromo-2,6-dimethylphenyl)-5-morpholino-2,6-dihydro-7H-[1,2,3]triazolo[4,5-d]pyrimidine-7-thione (27.1 mg, 64.3 mol, 5.22% yield, 99.7% purity) as a yellow solid.

¹H NMR: (400 MHz, DMSO-d6) δ 12.63-12.76 (m, 1H), 7.59 (s, 2H), 3.68-3.72 (m, 4H), 3.61-3.65 (m, 4H), 1.97 (s, 6H).

LC-MS: m/z=414.5 (M+H)⁺, Rt=2.754 min.

### Example 3: Preparation of 2-(4-cyclopropyl-2,6-dimethylphenyl)-5-morpholino-2,6-dihydro-7H-[1,2,3]triazolo[4,5-d]pyrimidin-7-one (Compound 3)

### Compound 3-1: morpholine-4-carboximidamide

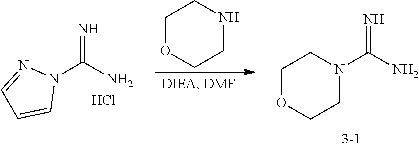 [SMILES: N=C(N)n1cccn1 ; Cl ; C1COCCN1 ; N=C(N)N1CCOCC1]

To a solution of pyrazole-1-carboxamidine (15 g, 102 mmol, HCL) in DMF (100 mL) was added DIEA (14.5 g, 113 mmol, 19.61 mL) and morpholine (8.92 g, 102 mmol, 9.01 mL). The mixture was stirred at 20° C. for 8 hr. TLC (CH₂Cl₂/MeOH=10/1) showed the reaction was completed. The reaction mixture was poured into methyl tertiary ether (1 L) and then white solid was precipitated, filtered and concentrated under reduced pressure to give morpholine-4-carboxamidine (14.0 g, crude) as a white solid.

¹H NMR (400 MHz, DMSO-d6): δ 7.72 (br s, 3H), 3.57-3.68 (m, 4H), 3.40-3.48 (m, 4H).

### Compound 3-2: 6-amino-2-morpholinopyrimidin-4(3H)-one

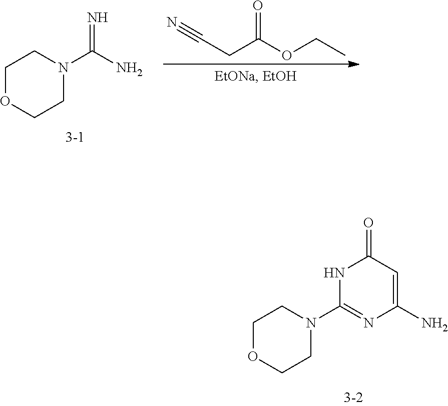

To a solution of morpholine-4-carboxamidine (14.0 g, 108 mmol) in EtOH (200 mL) was added EtONa (1 M, 200 mL) and ethyl 2-cyanoacetate (12.3 g, 108 mmol). The mixture was stirred at 20° C. for 8 hrs. LC-MS showed the reaction was completed. The reaction mixture was cooled to room temperature and filtered to give a residue, the residue was purified by column (CH₂Cl₂/MeOH=1/0 to 10/1) 4-amino-2-morpholino-1H-pyrimidin-6-one (14.0 g, crude) as a yellow solid.

LC-MS: m/z=197.2 (M+H)⁺, Rt=0.510 min.

¹H NMR (400 MHz, DMSO-d6): δ 10.25 (br s, 1H), 6.06 (s, 2H), 4.65 (s, 1H), 3.55-3.62 (m, 4H), 3.47-3.54 (m, 4H).

### Compound 3-3: 4-cyclopropyl-2,6-dimethylaniline

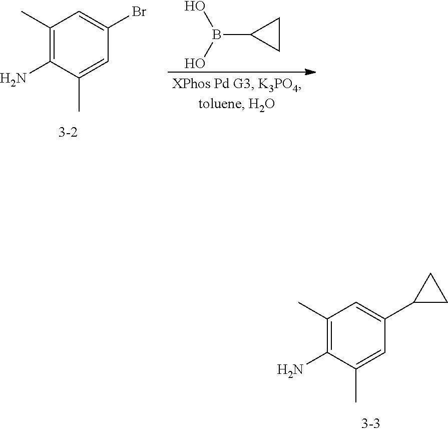

To a solution of 4-bromo-2,6-dimethyl-aniline (10.0 g, 50.0 mmol) and cyclopropylboronic acid (6.44 g, 74.9 mmol) in toluene (200 mL) and H₂O (50 mL) was added K₃PO₄ (31.8 g, 150 mmol) and XPhos Pd G3 (4.23 g, 5.00 mmol). The mixture was stirred at 110° C. for 8 hrs under N₂. LC-MS showed the reaction was completed. The reaction mixture was diluted with EtOAc (300 mL) and washed with H₂O (300 mL×2), dried over Na₂SO₄, filtered and concentrated under reduced pressure to give a residue. The residue was purified by column chromatography (SiO₂, Petroleum ether/Ethyl acetate=1/0 to 10/1) to give 4-cyclopropyl-2,6-dimethyl-aniline (4 g, 49.6% yield) as a brown oil.

LC-MS: m/z=162.1 (M+H)⁺, Rt=1.263 min.

¹H NMR (400 MHz, DMSO-d6): δ 6.53 (s, 2H), 4.26 (s, 2H), 2.03 (s, 6H), 1.61-1.75 (m, 1H), 0.68-0.82 (m, 2H), 0.41-0.52 (m, 2H).

### Compound 3-4: 2-(4-cyclopropyl-2,6-dimethylphenyl)-5-morpholino-1,2,3,6-tetrahydro-7H-[1,2,3]triazolo[4,5-d]pyrimidin-7-one

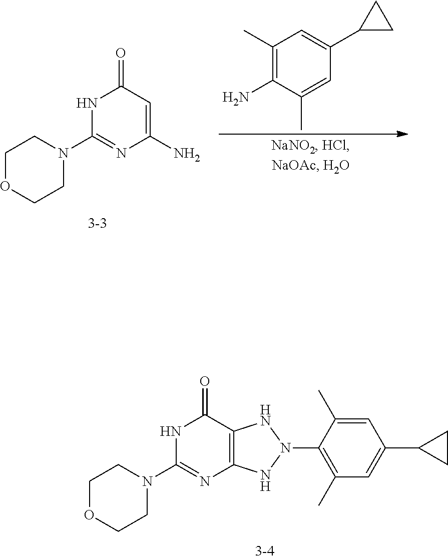 [SMILES: Nc1cc(=O)[nH]c(N2CCOCC2)n1 ; Cc1cc(C2CC2)cc(C)c1N ; Cc1cc(C2CC2)cc(C)c1N1Nc2nc(N3CCOCC3)[nH]c(=O)c2N1]

To a solution of 4-cyclopropyl-2,6-dimethyl-aniline (904 mg, 5.61 mmol) in HCl (10 mL) and H₂O (10 mL) was added NaNO₂ (387 mg, 5.61 mmol) at 0° C., after stirred for 30 min, 4-amino-2-morpholino-1H-pyrimidin-6-one (1 g, 5.1 mmol) and NaOAc (2.09 g, 25.5 mmol) was added. The mixture was stirred at 100° C. for 3 hrs. LC-MS showed the reaction was completed. The reaction mixture was filtered to give a residue. The residue was washed with H₂O (20 mL×2) and methyl tertiary butyl ether (20 mL×2) to give 2-(4-cyclopropyl-2,6-dimethyl-phenyl)-5-morpholino-3,6-dihydro-1H-triazolo[4,5-d]pyrimidin-7-one (1 g, crude) as a yellow solid.

LC-MS: m/z=369.2 (M+H)⁺, Rt=1.728 min.

### Compound 3: 2-(4-cyclopropyl-2,6-dimethylphenyl)-5-morpholino-2,6-dihydro-7H-[1,2,3]triazolo[4,5-d]pyrimidin-7-one

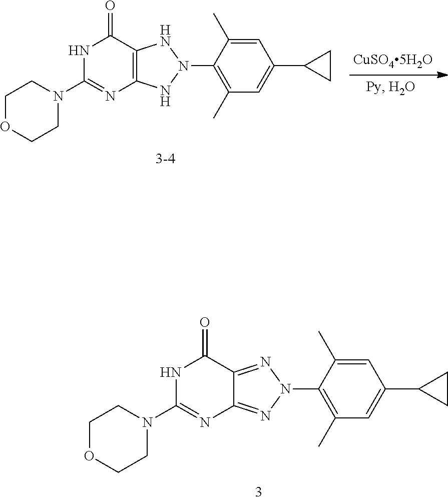 [SMILES: Cc1cc(C2CC2)cc(C)c1N1Nc2nc(N3CCOCC3)[nH]c(=O)c2N1 ; Cc1cc(C2CC2)cc(C)c1-n1nc2nc(N3CCOCC3)[nH]c(=O)c2n1]

To a solution of 2-(4-cyclopropyl-2,6-dimethyl-phenyl)-5-morpholino-3,6-dihydro-1H-triazolo[4,5-d]pyrimidin-7-one (900 mg, 2.44 mmol) in H₂O (10 mL) was added CuSO₄·5H₂O (1.22 g, 4.89 mmol, 2 eq) and Py (10 mL). The mixture was stirred at 100° C. for 5 hrs. LC-MS showed the reaction was completed. The reaction mixture was filtered to give a residue, the residue was washed with H₂O (20 mL×2) and then purified by prep-HPLC (column: Xtimate C18 150*40 mm*10 μm; mobile phase: [water(Nc₃H₂O+NH₄HCO₃)-ACN]; gradient: 6%-46% B over 25 min) to give 2-(4-cyclopropyl-2,6-dimethyl-phenyl)-5-morpholino-6H-triazolo[4,5-d]pyrimidin-7-one (500 mg, 55.83% yield, 99.94% purity) was obtained as a white solid.

LC-MS: m/z=367.2 (M+H)+, Rt=2.088 min.

¹H NMR (400 MHz, DMSO-d6): δ 11.45 (br s, 1H), 6.99 (s, 2H), 3.65-3.71 (m, 4H), 3.58-3.64 (m, 4H), 1.94-1.99 (m, 1H), 1.91 (s, 6H), 0.96-1.06 (m, 2H), 0.70-0.82 (m, 2H).

### Example 4: Preparation of 2-(4-cyclopropyl-2,6-dimethylphenyl)-5-morpholino-2,6-dihydro-7H-[1,2,3]triazolo[4,5-d]pyrimidine-7-thione (Compound 4)

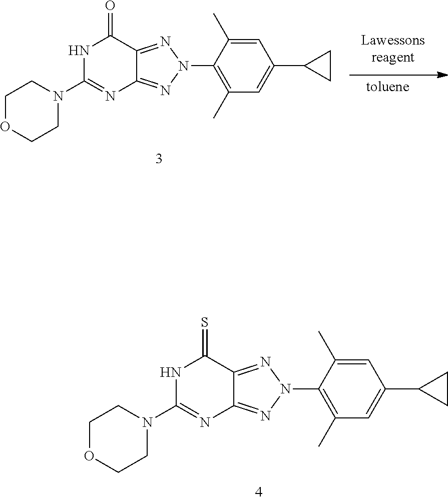 [SMILES: Cc1cc(C2CC2)cc(C)c1-n1nc2nc(N3CCOCC3)[nH]c(=O)c2n1 ; Cc1cc(C2CC2)cc(C)c1-n1nc2nc(N3CCOCC3)[nH]c(=S)c2n1]

To a solution of 2-(4-cyclopropyl-2,6-dimethyl-phenyl)-5-morpholino-6H-triazolo[4,5-d]pyrimidin-7-one (300 mg, 819 mol) in Tol. (10 mL) was added LAWESSON'S REAGENT (662 mg, 1.64 mmol). The mixture was stirred at 100° C. for 3 hrs. LC-MS showed the reaction was completed. The reaction mixture was concentrated under reduced pressure to give a residue. The residue was purified by prep-HPLC (column: Xtimate C18 150*40 mm*10 μm; mobile phase: [water(HCl)-ACN]; gradient: 0%-20% B over 20 min) to give 2-(4-cyclopropyl-2,6-dimethyl-phenyl)-5-morpholino-6H-triazolo-[4,5-d]pyrimidine-7-thione (25.66 mg, 8.16% yield, 99.6% purity) as a yellow solid.

LC-MS: m/z=383.3 (M+H)⁺, Rt=5.467 min.

1H NMR (400 MHz, DMSO-d6): δ 12.66 (br s, 1H), 6.99 (s, 2H), 3.66-3.74 (m, 4H), 3.59-3.66 (m, 4H), 1.86-2.02 (m, 7H), 0.96-1.06 (m, 2H), 0.71-0.81 (m, 2H).

### Examples 5-50

Compounds 5-50 can be prepared following one of the procedures described above in Examples 1-4 using commercially available starting materials and agents.

### Example 51: Preparation of 2-(4-bromo-2,6-dimethylphenyl)-5-(pyrrolidin-1-yl)-2,6-dihydro-7H-[1,2,3]triazolo[4,5-d]pyrimidin-7-one (Compound 51)

### Compound 51-1: pyrrolidine-1-carboximidamide

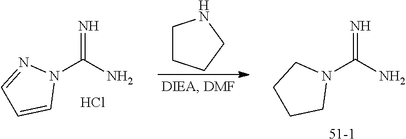

To a solution of pyrazole-1-carboxamidine (15 g, 102 mmol, HCl) in DMF (200 mL) was added DIEA (14.5 g, 113 mmol, 19.6 mL) and pyrrolidine (7.28 g, 102 mmol, 8.54 mL). The mixture was stirred at 20° C. for 8 hrs. TLC (CH2Cl2/MeOH=5/1) showed the reaction was completed. The reaction mixture was poured into methyl tertiary ether (1 L) and then white solid was precipitated, filtered and concentrated under reduced pressure to give pyrrolidine-1-carboximidamide (13.0 g, crude) as a white solid.

¹H NMR (400 MHz, DMSO-d6): δ 7.42 (br s, 4H), 3.28-3.35 (m, 4H), 1.83-1.94 (m, 4H).

### Compound 51-2: 6-amino-2-(pyrrolidin-1-yl)pyrimidin-4(3H)-one

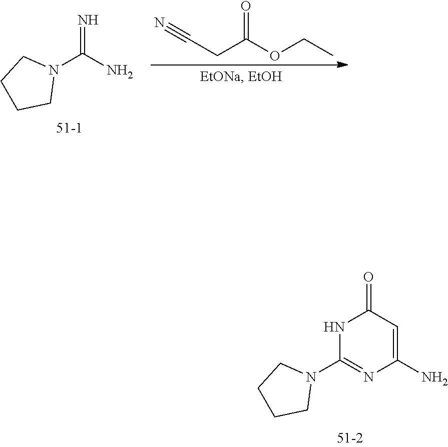

To a solution of pyrrolidine-1-carboxamidine (13 g, 115 mmol) in EtOH (500 mL) was added NaOEt (1 M, 500 mL) and ethyl 2-cyanoacetate (12.9 g, 115 mmol). The mixture was stirred at 80° C. for 8 hrs. TLC (CH2Cl2/MeOH=10/1) showed the reaction was completed. The reaction mixture was cooled to room temperature and filtered to give a residue, the residue was purified by column (CH2Cl2/MeOH=1/0 to 10/1) to give 4-amino-2-pyrrolidin-1-yl-1H-pyrimidin-6-one (15.0 g, 72.5% yield) as a yellow solid.

¹H NMR (400 MHz, DMSO-d6): δ 9.89 (br s, 1H), 5.92 (s, 2H), 4.49 (s, 1H), 3.35-3.39 (m, 4H), 1.80-1.88 (m, 4H).

### Compound 51-3: 2-(4-bromo-2,6-dimethylphenyl)-5-(pyrrolidin-1-yl)-1,2,3,6-tetrahydro-7H-[1,2,3]triazolo[4,5-d]pyrimidin-7-one

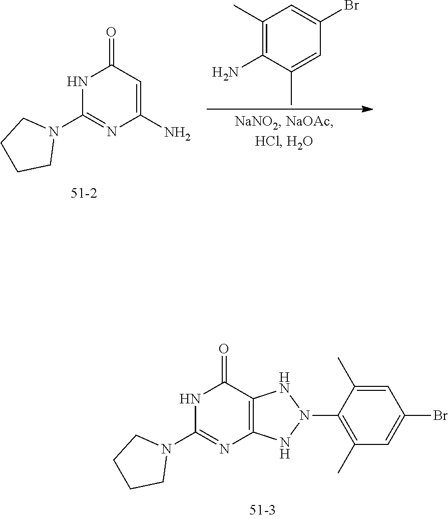

To a solution of 4-bromo-2,6-dimethyl-aniline (1.11 g, 5.55 mmol) in HCl (10 mL) and H₂O (10 mL) was added NaNO₂ (421 mg, 6.1 mmol) at 0° C., after stirred for 30 min, 4-amino-2-morpholino-1H-pyrimidin-6-one (1 g, 5.1 mmol) and NaOAc (2.09 g, 25.5 mmol) was added. The mixture was stirred at 100° C. for 3 hrs. LC-MS showed the reaction was completed. The reaction mixture was filtered to give a residue. The residue was washed with H₂O (20 mL) for 3 times to give 2-(4-bromo-2,6-dimethyl-phenyl)-5-pyrrolidin-1-yl-3,6-dihydro-1H-triazolo[4,5-d]pyrimidin-7-one (900 mg, 41.5% yield) as a yellow solid.

LC-MS: m/z=391.0 (M+H)⁺, Rt=1.671 min.

### Compound 51: 2-(4-bromo-2,6-dimethylphenyl)-5-(pyrrolidin-1-yl)-2,6-dihydro-7H-[1,2,3]triazolo[4,5-d]pyrimidin-7-one

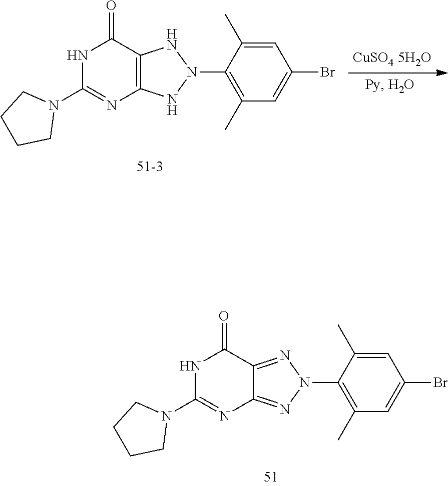 [SMILES: Cc1cc(Br)cc(C)c1N1Nc2nc(N3CCCC3)[nH]c(=O)c2N1 ; Cc1cc(Br)cc(C)c1-n1nc2nc(N3CCCC3)[nH]c(=O)c2n1]

To a solution of 2-(4-bromo-2,6-dimethyl-phenyl)-5-pyrrolidin-1-yl-3,6-dihydro-1H-triazolo[4,5-d]pyrimidin-7-one (900 mg, 2.3 mmol) in H₂O (10 mL) was added CuSO₄·5H₂O (1.15 g, 4.6 mmol) and Py (10 mL). The mixture was stirred at 100° C. for 2 hrs. LC-MS showed the reaction was completed. The reaction mixture was filtered to give a residue. The residue was washed with H₂O (30 mL×3) and tert-butyl methyl ether (30 mL×3) to give 2-(4-bromo-2,6-dimethyl-phenyl)-5-pyrrolidin-1-yl-6H-triazolo[4,5-d]pyrimidin-7-one (650 mg, 72.1% yield, 99.2% purity) as a yellow solid.

LC-MS: m/z=389.1 (M+H)+, Rt=2.11 min.

¹H NMR (400 MHz, DMSO-d6): δ 11.15 (s, 1H), 7.57 (s, 2H), 3.48-3.54 (m, 4H), 1.96 (s, 6H), 1.89-1.94 (m, 4H).

### Example 52: Preparation of 2-(4-bromo-2,6-dimethylphenyl)-5-(pyrrolidin-1-yl)-2,6-dihydro-7H-[1,2,3]triazolo[4,5-d]pyrimidine-7-thione (Compound 52)

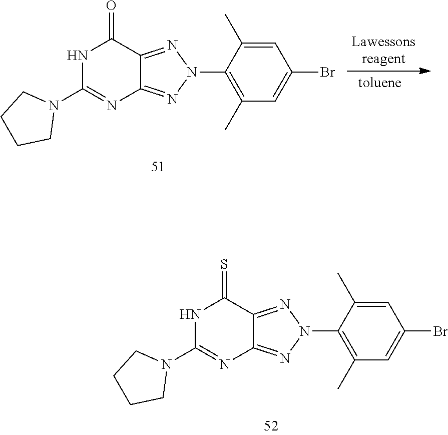 [SMILES: Cc1cc(Br)cc(C)c1-n1nc2nc(N3CCCC3)[nH]c(=O)c2n1 ; Cc1cc(Br)cc(C)c1-n1nc2nc(N3CCCC3)[nH]c(=S)c2n1]

To a solution of 2-(4-bromo-2,6-dimethyl-phenyl)-5-pyrrolidin-1-yl-6H-triazolo[4,5-d]pyrimidin-7-one (200 mg, 514 mol) in toluene (5 mL) was added LAWESSON'S REAGENT (623 mg, 1.54 mmol). The mixture was stirred at 100° C. for 2 hrs. LC-MS showed the reaction was completed. The reaction mixture was concentrated under reduced pressure to give a residue. The residue was purified by prep-HPLC (column: Xtimate C18 150*40 mm*10 μm; mobile phase: [water(HCl)-ACN]; gradient: 34%-74% B over 25 min) to give 2-(4-bromo-2,6-dimethyl-phenyl)-5-pyrrolidin-1-yl-6H-triazolo[4,5-d]pyrimidine-7-thione (26.16 mg, 12.4% yield, 99.03% purity) as a yellow solid.

LC-MS: m/z=407.1 (M+2+H)+, Rt=5.928 min.

¹H NMR (400 MHz, DMSO-d6): δ 11.64-12.46 (m, 1H), 7.59 (s, 2H), 3.57-3.63 (m, 4H), 1.98 (s, 6H), 1.91-1.96 (m, 4H).

### Example 53: Preparation of 2-(4-cyclopropyl-2,6-dimethylphenyl)-5-(pyrrolidin-1-yl)-2,6-dihydro-7H-[1,2,3]triazolo[4,5-d]pyrimidin-7-one (Compound 53)

### Compound 53-1: 2-(4-cyclopropyl-2,6-dimethylphenyl)-5-(pyrrolidin-1-yl)-1,2,3,6-tetrahydro-7H-[1,2,3]triazolo[4,5-d]pyrimidin-7-one

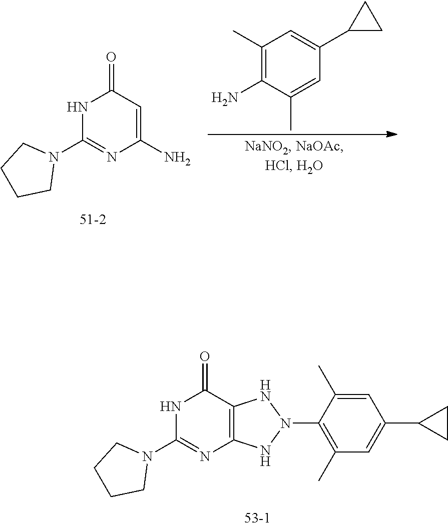 [SMILES: Nc1cc(=O)[nH]c(N2CCCC2)n1 ; Cc1cc(C2CC2)cc(C)c1N ; Cc1cc(C2CC2)cc(C)c1N1Nc2nc(N3CCCC3)[nH]c(=O)c2N1]

To a solution of 4-cyclopropyl-2,6-dimethylaniline (895 mg, 5.55 mmol) in HCl (10 mL) and H₂O (10 mL) was added (421 mg, 6.1 mmol) at 0° C., after stirred for 30 min, 4-amino-2-pyrrolidin-1-yl-1H-pyrimidin-6-one (1 g, 5.55 mmol) and NaOAc (2.28 g, 27.7 mmol) was added. The mixture was stirred at 100° C. for 3 hrs. LC-MS showed the reaction was completed. The reaction mixture was filtered to give a residue. The residue was washed with H₂O (20 mL×2) and methyl tertiary butyl ether (20 mL×2) to give 2-(4-cyclopropyl-2,6-dimethyl-phenyl)-5-pyrrolidin-1-yl-3,6-dihydro-1H-triazolo[4,5-d]pyrimidin-7-one (1 g, crude) as a yellow solid.

LC-MS: m/z=353.2 (M+H)+, Rt=1.833 min.

### Compound 53: 2-(4-cyclopropyl-2,6-dimethylphenyl)-5-(pyrrolidin-1-yl)-2,6-dihydro-7H-[1,2,3]triazolo[4,5-d]pyrimidin-7-one

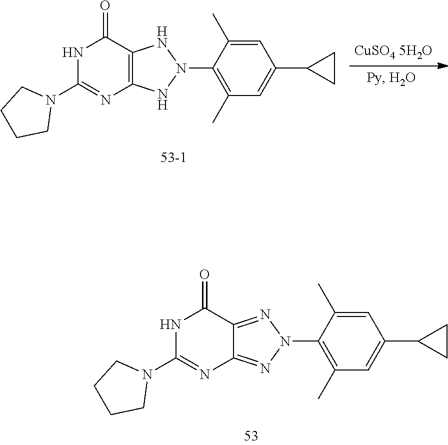 [SMILES: Cc1cc(C2CC2)cc(C)c1N1Nc2nc(N3CCCC3)[nH]c(=O)c2N1 ; Cc1cc(C2CC2)cc(C)c1-n1nc2nc(N3CCCC3)[nH]c(=O)c2n1]

To a solution of 2-(4-cyclopropyl-2,6-dimethyl-phenyl)-5-pyrrolidin-1-yl-3,6-dihydro-1H-triazolo[4,5-d]pyrimidin-7-one (1 g, 2.84 mmol) in H₂O (10 mL) was added CuSO₄·5H₂O (1.42 g, 5.67 mmol) and Py (10 mL). The mixture was stirred at 100° C. for 5 hrs. LC-MS showed the reaction was completed. The reaction mixture was filtered to give a residue. The residue was purified by prep-HPLC (column: Xtimate C18 150*40 mm*10 μm; mobile phase: [water(NH₃H₂O+NH₄HCO₃)-ACN]; gradient: 24%-64% B over 25 min) to give 2-(4-cyclopropyl-2,6-dimethyl-phenyl)-5-pyrrolidin-1-yl-6H-triazolo[4,5-d]pyrimidin-7-one (27.27 mg, 99.69% purity) as a white solid.

LC-MS: m/z=351.2 (M+H)+, Rt=2.135 min.

¹H NMR: (400 MHz, DMSO-d6)

δ 10.84-11.27 (m, 1H), 6.98 (s, 2H), 3.48-3.54 (m, 4H), 1.89-1.99 (m, 11H), 0.97-1.04 (m, 2H), 0.73-0.80 (m, 2H).

### Example 54: Preparation of 2-(4-cyclopropyl-2,6-dimethylphenyl)-5-(pyrrolidin-1-yl)-2,6-dihydro-7H-[1,2,3]triazolo[4,5-d]pyrimidine-7-thione (Compound 54)

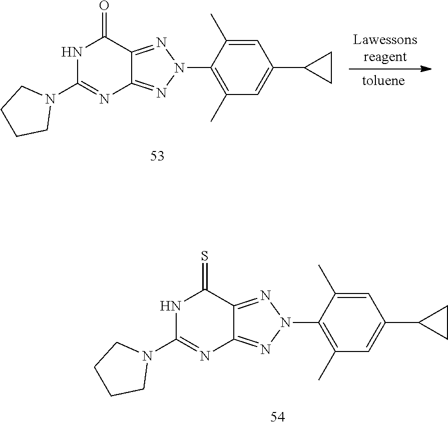 [SMILES: Cc1cc(C2CC2)cc(C)c1-n1nc2nc(N3CCCC3)[nH]c(=O)c2n1 ; Cc1cc(C2CC2)cc(C)c1-n1nc2nc(N3CCCC3)[nH]c(=S)c2n1]

To a solution of 2-(4-cyclopropyl-2,6-dimethyl-phenyl)-5-pyrrolidin-1-yl-6H-triazolo[4,5-d]pyrimidin-7-one (200 mg, 571 mol) in toluene (5 mL) was added LAWESSON'S REAGENT (462 mg, 1.14 mmol). The mixture was stirred at 100° C. for 2 hrs. LC-MS showed the reaction was completed. The reaction mixture was concentrated under reduced pressure to give a residue. The residue was purified by prep-HPLC (column: Xtimate C18 150*40 mm*10 μm; mobile phase: [water(TFA)-ACN]; gradient: 32%-72% B over 25 min) to give 2-(4-cyclopropyl-2,6-dimethyl-phenyl)-5-pyrrolidin-1-yl-6H-triazolo[4,5-d]pyrimidine-7-thione (29.24 mg, 13.95% yield, 99.79% purity) as a yellow solid.

LC-MS: m/z=367.3 (M+H)⁺, Rt=5.937 min.

¹H NMR: (400 MHz, DMSO-d6)

δ 12.12 (s, 1H), 6.99 (s, 2H), 3.56-3.63 (m, 4H), 1.91-1.98 (m, 11H), 0.97-1.05 (m, 2H), 0.73-0.81 (m, 2H).

### Example 61: Preparation of 2-(2,6-dimethyl-4-(trifluoromethyl)phenyl)-5-(pyrrolidin-1-yl)-2,6-dihydro-7H-[1,2,3]triazolo[4,5-d]pyrimidin-7-one (Compound 61)

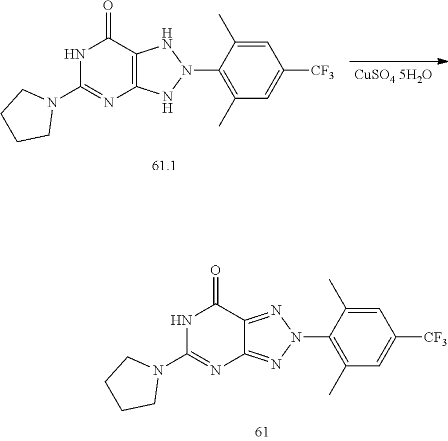 [SMILES: Cc1cc(C(F)(F)F)cc(C)c1N1Nc2nc(N3CCCC3)[nH]c(=O)c2N1 ; Cc1cc(C(F)(F)F)cc(C)c1-n1nc2nc(N3CCCC3)[nH]c(=O)c2n1]

To a solution of 2-(2,6-dimethyl-4-(trifluoromethyl)phenyl)-5-(pyrrolidin-1-yl)-1,2,3,6-tetrahydro-7H-[1,2,3]triazolo[4,5-d]pyrimidin-7-one (450 mg, 1.18 mmol), CuSO4·5H2O (590 mg, 2.37 mmol) in Pyridine (4 mL), H2O (4 mL) was degassed and purged with N2 for 3 times, and then the mixture was stirred at 100° C. for 10 hrs under N2 atmosphere. LC-MS showed the reaction was completed. The reaction mixture was filtered to give a residue. The residue was washed with H2O (30 mL×2) and methyl tertiary butyl ether (30 mL×2) to give 2-(2,6-dimethyl-4-(trifluoromethyl)phenyl)-5-(pyrrolidin-1-yl)-2,6-dihydro-7H-[1,2,3]triazolo[4,5-d]pyrimidin-7-one (298 mg, 66.2% yield, 99.4% purity) as a yellow solid.

LCMS: m/z=379.0 (M+H)⁺, Rt=2.195 min

1H NMR: (400 MHz, DMSO-d6)

δ 10.96-11.38 (m, 1H), 7.60-7.88 (m, 2H), 3.48-3.68 (m, 4H), 2.06 (s, 6H), 1.92 (s, 4H)

### Example 63: Preparation of 2-(4-chloro-2,6-dimethylphenyl)-5-(pyrrolidin-1-yl)-2,6-dihydro-7H-[1,2,3]triazolo[4,5-d]pyrimidin-7-one (Compound 63)

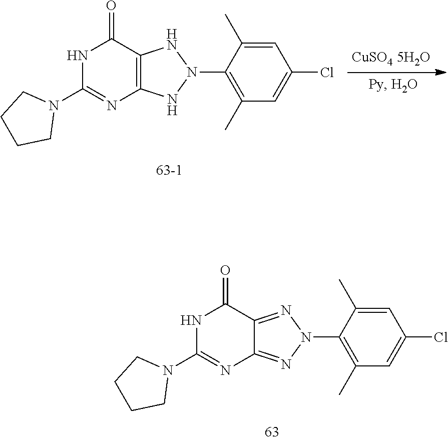 [SMILES: Cc1cc(Cl)cc(C)c1N1Nc2nc(N3CCCC3)[nH]c(=O)c2N1 ; Cc1cc(Cl)cc(C)c1-n1nc2nc(N3CCCC3)[nH]c(=O)c2n1]

To a solution of 2-(4-chloro-2,6-dimethylphenyl)-5-(pyrrolidin-1-yl)-1,2,3,6-tetrahydro-7H-[1,2,3]triazolo[4,5-d]pyrimidin-7-one (900 mg, 2.60 mmol), CuSO4·5H2O (1.30 g, 5.19 mmol) in Py (10 mL) and H2O (10 mL) was degassed and purged with N2 for 3 times, and then the mixture was stirred at 100° C. for 10 hrs under N2 atmosphere. LCMS (EB5915-351-P1A) showed the reaction was completed. The reaction mixture was filtered to give a residue. The residue was washed with H2O (30 mL×2) and methyl tertiary butyl ether (30 mL×2) to give 2-(4-chloro-2,6-dimethylphenyl)-5-(pyrrolidin-1-yl)-2,6-dihydro-7H-[1,2,3]triazolo[4,5-d]pyrimidin-7-one (560 mg, 62.1% yield, 99.2% purity) as a yellow solid.

LCMS: m/z=367.2 (M+H)+, Rt=2.088 min

1H NMR: (400 MHz, DMSO-d6)

δ 11.16 (s, 1H), 7.43 (s, 2H), 3.51 (t, J=6.4 Hz, 4H), 1.96 (s, 6H), 1.90-1.94 (m, 4H)

### Example 75: Preparation of 2-(4-bromo-2-(trifluoromethyl)phenyl)-5-(pyrrolidin-1-yl)-2,6-dihydro-7H-[1,2,3]triazolo[4,5-d]pyrimidin-7-one (Compound 75)

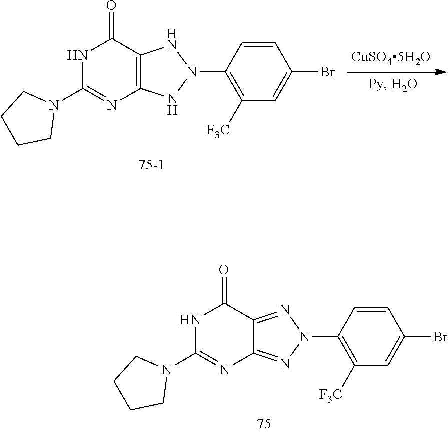 [SMILES: O=c1[nH]c(N2CCCC2)nc2c1NN(c1ccc(Br)cc1C(F)(F)F)N2 ; O=c1[nH]c(N2CCCC2)nc2nn(-c3ccc(Br)cc3C(F)(F)F)nc12]

CuSO4·5H2O (1.13 g, 4.52 mmol) was dissolved in Py (20 mL) and H2O (20 mL), and the resulting mixture was heated to 100° C., 2-[4-bromo-2-(trifluoromethyl)phenyl]-5-pyrrolidin-1-yl-3,6-dihydro-1H-triazolo[4,5-d]pyrimidin-7-one (650 mg, 1.51 mmol) was carefully added to the hot solution. Then mixture was stirred at 100° C. for 10 hrs. TLC (Petroleum ether/EtOAc=1/1, UV) showed Reactant 1 was consumed completely. The reaction mixture was filtered to give a residue. The residue was washed with H2O (30 mL×3) and MeOH (10 mL×3) to give 2-(4-bromo-2-(trifluoromethyl)phenyl)-5-(pyrrolidin-1-yl)-2,6-dihydro-7H-[1,2,3]triazolo[4,5-d]pyrimidin-7-one (240 mg, 35.9% yield, 96.9% purity) as a yellow solid.

1H NMR: (400 MHz, DMSO-d6)

δ 11.22 (s, 1H), 8.25 (s, 1H), 8.17 (d, J=8.4 Hz, 1H), 7.86 (d, J=8.6 Hz, 1H), 3.52 (s, 4H), 1.92 (s, 4H)

### Example 77: Preparation of 2-(4-cyclopropyl-2-(trifluoromethyl)phenyl)-5-(pyrrolidin-1-yl)-2,6-dihydro-7H-[1,2,3]triazolo[4,5-d]pyrimidin-7-one (Compound 77)

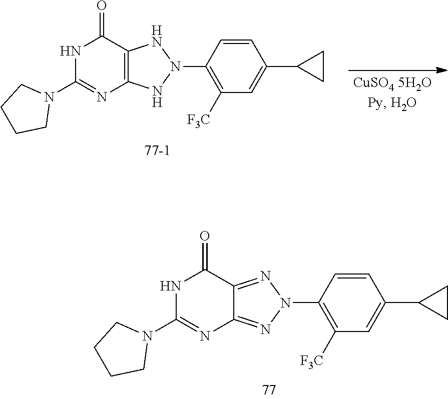 [SMILES: O=c1[nH]c(N2CCCC2)nc2c1NN(c1ccc(C3CC3)cc1C(F)(F)F)N2 ; O=c1[nH]c(N2CCCC2)nc2nn(-c3ccc(C4CC4)cc3C(F)(F)F)nc12]

To a solution of 2-(4-cyclopropyl-2-(trifluoromethyl)phenyl)-5-(pyrrolidin-1-yl)-1,2,3,6-tetrahydro-7H-[1,2,3]triazolo[4,5-d]pyrimidin-7-one (500 mg, 1.27 mmol) in Py (5.00 mL) and H2O (5.00 mL) was added CuSO4·5H2O (954.51 mg, 3.82 mmol). The mixture was stirred at 100° C. for 10 hrs. LCMS(EB12552-14-P1A1) show the reaction was completed showed the reaction was completed. The reaction mixture was filtered to give a residue, then the residue was washed with H2O (20.0 mL×2) and methanol (5.00 ml) to give 2-(4-cyclopropyl-2-(trifluoromethyl)phenyl)-5-(pyrrolidin-1-yl)-2,6-dihydro-7H-[1,2,3]triazolo[4,5-d]pyrimidin-7-one (115 mg, 22.3% yield, 96.5% purity) as a yellow solid.

LCMS: m/z=409.0 (M+H)+, Rt=1.534 min

1H NMR: (400 MHz, DMSO-d6)

δ 12.66 (s, 1H), 6.99 (s, 2H), 3.66-3.74 (m, 4H), 3.59-3.66 (m, 4H), 1.86-2.02 (m, 7H), 0.96-1.06 (m, 2H), 0.71-0.81 (m, 2H)

### Example 91: Preparation of 2-(4-bromo-2,6-dichlorophenyl)-5-(pyrrolidin-1-yl)-2,6-dihydro-7H-[1,2,3]triazolo[4,5-d]pyrimidin-7-one (Compound 91)

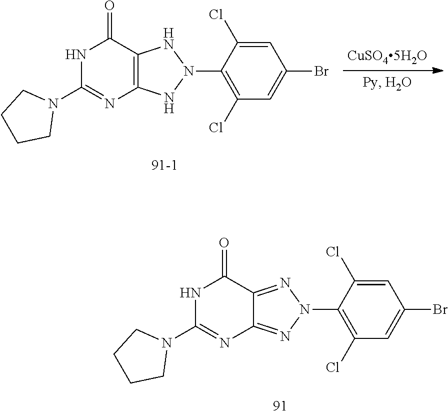 [SMILES: O=c1[nH]c(N2CCCC2)nc2c1NN(c1c(Cl)cc(Br)cc1Cl)N2 ; O=c1[nH]c(N2CCCC2)nc2nn(-c3c(Cl)cc(Br)cc3Cl)nc12]

CuSO4·5H2O (867 mg, 3.47 mmol) was dissolved in Py (20 mL) and H2O (20 mL), and the resulting mixture was heated to 100° C., 2-(4-bromo-2,6-dichloro-phenyl)-5-pyrrolidin-1-yl-3,6-dihydro-1H-triazolo[4,5-d]pyrimidin-7-one (500 mg, 1.16 mmol) was carefully added to the hot solution. Turn the reaction mixture dark green. Then the mixture was 100° C. for 10 hrs. LC-MS showed Reactant 1 was consumed completely. The reaction mixture was filtered to give a cake and washed with H2O (20 mL×5) to give a residue, which was purified by prep-HPLC (column: Welch Xtimate C18 40*200 mm 7 um; mobile phase: [water(NH3H2O+NH4HCO3)-MeOH]; gradient: 38%-78% B over 25 min) to give 2-(4-bromo-2,6-dichlorophenyl)-5-(pyrrolidin-1-yl)-2,6-dihydro-7H-[1,2,3]triazolo[4,5-d]pyrimidin-7-one 2-(4-bromo-2,6-dichloro-phenyl)-5-pyrrolidin-1-yl-6H-triazolo[4,5-d]pyrimidin-7-one (50.4 mg, 9.77% yield, 96.28% purity) as a white solid.

LCMS: m/z=428.7, 430.7 (M+H, M+H+2)+, Rt=2.053 min

1H NMR: (400 MHz, DMSO-d6)

δ 11.26 (br s, 1H), 8.18 (s, 2H), 3.52 (br t, J=6.4 Hz, 4H), 1.92 (br t, J=6.4 Hz, 4H)

### Example 95: Preparation of 2-(4-bromo-2,6-dimethylphenyl)-5-(diethylamino)-2,6-dihydro-7H-[1,2,3]triazolo[4,5-d]pyrimidin-7-one (Compound 95)

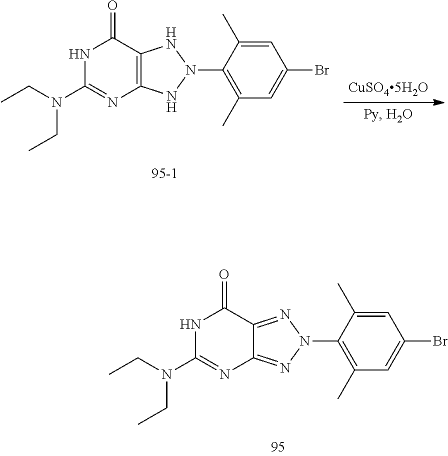 [SMILES: CCN(CC)c1nc2c(c(=O)[nH]1)NN(c1c(C)cc(Br)cc1C)N2 ; CCN(CC)c1nc2nn(-c3c(C)cc(Br)cc3C)nc2c(=O)[nH]1]

CuSO4·5H2O (419 mg, 1.68 mmol) was dissolved in Py (3.00 mL) and H2O (3.00 mL), and the resulting mixture was heated to 100° C., 2-(4-bromo-2,6-dimethyl-phenyl)-5-(diethylamino)-3,6-dihydro-1H-triazolo[4,5-d]pyrimidin-7-one (220 mg, 559 μmol) was carefully added to the hot solution. Then the mixture was stirred at 100° C. for 10 hrs. TLC (CH2Cl2/MeOH=15/1, UV) showed Reactant 1 was consumed completely. The reaction mixture was filtered to give a residue. The residue was washed with H2O (10 mL×3) and MeOH (10 mL×3) to give 2-(4-bromo-2,6-dimethylphenyl)-5-(diethylamino)-2,6-dihydro-7H-[1,2,3]triazolo[4,5-d]pyrimidin-7-one (55.7 mg, 25.2% yield, 99.2% purity) as a brown solid.

1H NMR: (400 MHz, DMSO-d6)

δ 11.09 (s, 1H), 7.56 (s, 2H), 3.53-3.60 (m, 4H), 1.96 (s, 6H), 1.14 (t, J=6.8 Hz, 6H)

LCMS: m/z=393.1 (M+H+2)+, Rt=2.501 min

### Example 96: Preparation of 5-(azetidin-1-yl)-2-(4-bromo-2,6-dimethylphenyl)-2,6-dihydro-7H-[1,2,3]triazolo[4,5-d]pyrimidin-7-one (Compound 96)

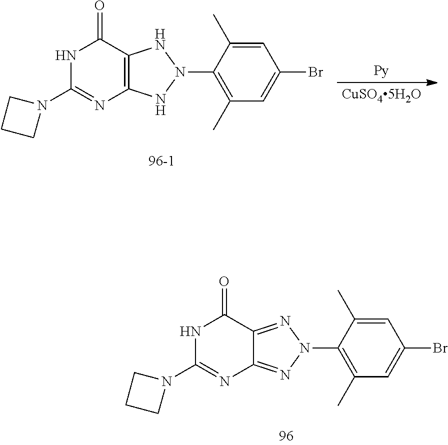 [SMILES: Cc1cc(Br)cc(C)c1N1Nc2nc(N3CCC3)[nH]c(=O)c2N1 ; Cc1cc(Br)cc(C)c1-n1nc2nc(N3CCC3)[nH]c(=O)c2n1]

To a solution of 5-(azetidin-1-yl)-2-(4-bromo-2,6-dimethyl-phenyl)-3,6-dihydro-1H-triazolo[4,5-d]pyrimidin-7-one (200 mg, 530 mol) in Py (2 mL)/H2O (2 mL) was added CuSO4·5H2O (264 mg, 1.06 mmol). The mixture was stirred at 100° C. for 12 hrs. LCMS showed 93.3% of desired compound was detected. The reaction mixture was adjusted with water (10 ml) solution then filtered. The filtered cake was dried under vacuum pump. The crude product was triturated with MeOH (5 ml) at 25° C. for 10 min to give 5-(azetidin-1-yl)-2-(4-bromo-2,6-dimethyl-phenyl)-6H-triazolo[4,5-d]pyrimidin-7-one (23.0 mg, 11.4% yield, 98.5% purity) as a yellow solid.

LCMS: m/z=375.0, 377.1 (M+H, M+2)+, Rt=1.958 min

1H NMR: EB9426-122-P1D (400 MHz, DMSO-d6)

δ 11.38-11.69 (m, 1H), 7.35-7.74 (m, 2H), 3.96-4.27 (m, 4H), 2.21-2.37 (m, 2H), 1.83-2.06 (m, 6H)

### Example 97: Preparation of 2-(4-bromo-2-chlorophenyl)-5-(pyrrolidin-1-yl)-2,6-dihydro-7H-[1,2,3]triazolo[4,5-d]pyrimidin-7-one (Compound 97)

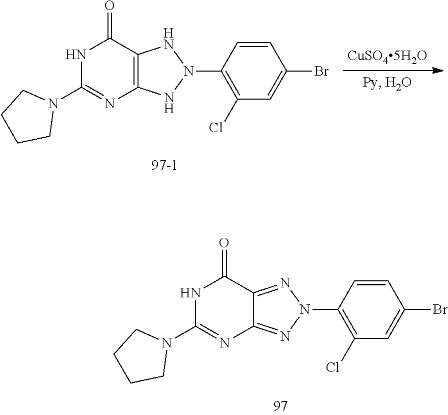 [SMILES: O=c1[nH]c(N2CCCC2)nc2c1NN(c1ccc(Br)cc1Cl)N2 ; O=c1[nH]c(N2CCCC2)nc2nn(-c3ccc(Br)cc3Cl)nc12]

CuSO4·5H2O (1.38 g, 5.51 mmol) was dissolved in Py (20.0 mL) and H2O (20.0 mL), and the resulting mixture was heated to 100° C., 2-(4-bromo-2,6-dichloro-phenyl)-5-pyrrolidin-1-yl-3,6-dihydro-1H-triazolo[4,5-d]pyrimidin-7-one (500 mg, 1.16 mmol) was carefully added to the hot solution. Then the mixture was stirred at 100° C. for 10 hrs. TLC (Petroleum ether/EtOAc=1/1, UV) showed Reactant 1 was consumed completely. The reaction mixture was filtered to give a residue. The residue was washed with H2O (30 mL×3) and MeOH (10 mL×3) to give 2-(4-bromo-2-chlorophenyl)-5-(pyrrolidin-1-yl)-2,6-dihydro-7H-[1,2,3]triazolo[4,5-d]pyrimidin-7-one (471 mg, 64.1% yield, 98.8% purity) as a yellow solid.

1H NMR: (400 MHz, DMSO-d6)

δ 11.20 (s, 1H), 8.09 (d, J=1.6 Hz, 1H), 7.73-7.85 (m, 2H), 3.47-3.56 (m, 4H), 1.92 (s, 4H)

LCMS: m/z=397.0 (M+H+2)+, Rt=2.114 min

### Example 98: Preparation of 2-(4-bromo-2-chloro-6-fluorophenyl)-5-(pyrrolidin-1-yl)-2,6-dihydro-7H-[1,2,3]triazolo[4,5-d]pyrimidin-7-one (Compound 98)

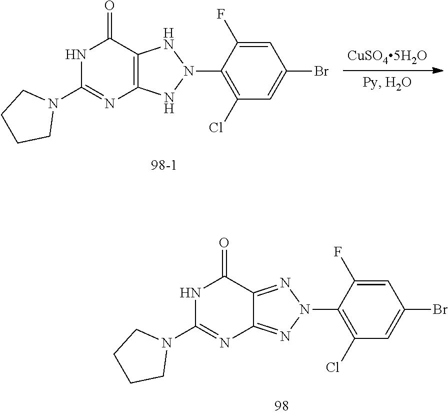 [SMILES: O=c1[nH]c(N2CCCC2)nc2c1NN(c1c(F)cc(Br)cc1Cl)N2 ; O=c1[nH]c(N2CCCC2)nc2nn(-c3c(F)cc(Br)cc3Cl)nc12]

CuSO4·5H2O (1.35 g, 5.41 mmol) was dissolved in Py (20 mL) and H2O (20 mL), and the resulting mixture was heated to 100° C. 2-(4-bromo-2-chloro-6-fluorophenyl)-5-(pyrrolidin-1-yl)-1,2,3,6-tetrahydro-7H-[1,2,3]triazolo[4,5-d]pyrimidin-7-one (750 mg, 1.80 mmol) was carefully added to the hot solution. Turn the reaction mixture dark green. Then the mixture was 100° C. for 10 hrs. LCMS showed reaction was completed. The reaction mixture was filtered to give a cake and washed with H2O (20 mL×5) to give a residue, which was purified by prep-HPLC (column: Welch Xtimate C18 40*200 mm 7 um; mobile phase: [water(NH3H2O+NH4HCO3)-MeOH]; gradient: 38%-78% B over 25 min) to give 2-(4-bromo-2-chloro-6-fluorophenyl)-5-(pyrrolidin-1-yl)-2,6-dihydro-7H-[1,2,3]triazolo[4,5-d]pyrimidin-7-one (55.4 mg, 7.33% yield, 98.69% purity) as a white solid

LCMS: m/z=412.8, 414.8 (M+H, M+H+2)+, Rt=1.930 min

1H NMR: (400 MHz, DMSO-d6)

δ 10.73-11.42 (m, 1H), 8.03 (s, 1H), 7.93-8.00 (m, 1H), 3.52 (br s, 4H), 1.92 (br s, 4H)

### Example 99: Preparation of 2-(4-bromo-2,6-difluorophenyl)-5-(pyrrolidin-1-yl)-2,6-dihydro-7H-[1,2,3]triazolo[4,5-d]pyrimidin-7-one (Compound 99)

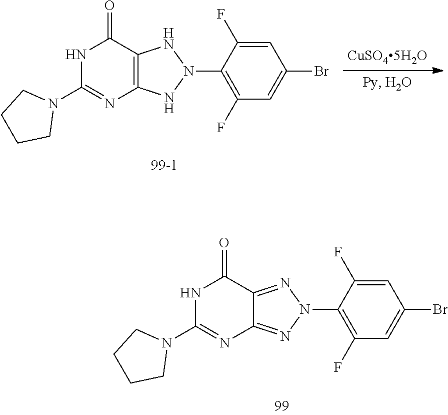 [SMILES: O=c1[nH]c(N2CCCC2)nc2c1NN(c1c(F)cc(Br)cc1F)N2 ; O=c1[nH]c(N2CCCC2)nc2nn(-c3c(F)cc(Br)cc3F)nc12]

CuSO4·5H2O (1.44 g, 5.79 mmol) was dissolved in Py (20 mL) and H2O (20 mL), and the resulting mixture was heated to 75° C. 2-(4-bromo-2,6-difluoro-phenyl)-5-pyrrolidin-1-yl-3,6-dihydro-1H-triazolo[4,5-d]pyrimidin-7-one (770 mg, 1.93 mmol) was carefully added to the hot solution. Turn the reaction mixture dark green. Then the mixture was stirred 100° C. for 10 hrs. LCMS showed Reactant 1 was consumed completely. The reaction mixture was filtered to give a cake, which was washed with H2O (20 mL×5) and MeOH (20 mL×3) to give 2-(4-bromo-2,6-difluoro-phenyl)-5-pyrrolidin-1-yl-6H-triazolo[4,5-d]pyrimidin-7-one (82.5 mg, 10.3% yield, 95.96% purity) as a yellow solid.

LCMS: m/z=396.9, 398.9 (M+H)+, Rt=1.853 min

1H NMR: (400 MHz, DMSO-d6)

δ 11.27 (br s, 1H), 7.93 (d, J=8.0 Hz, 2H), 3.51 (br t, J=6.0 Hz, 4H), 1.92 (br s, 4H)

### Example 100: Preparation of 2-(4-cyclopropyl-2-(trifluoromethyl)phenyl)-5-(4-ethylpiperazin-1-yl)-2,6-dihydro-7H-[1,2,3]triazolo[4,5-d]pyrimidin-7-one (Compound 100)

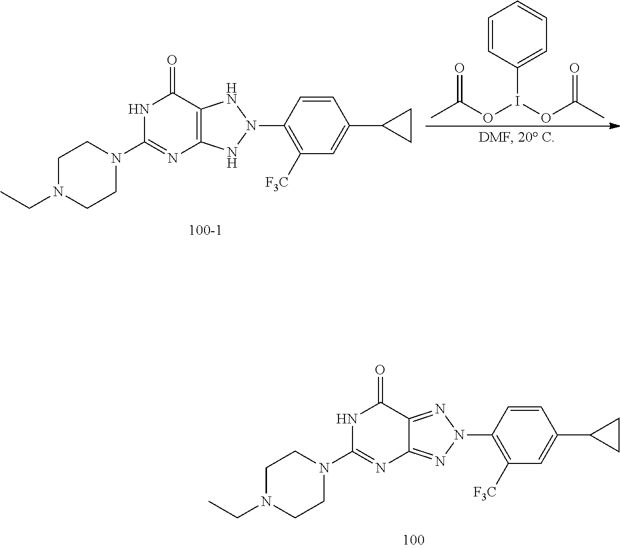

To a solution of 2-[4-cyclopropyl-2-(trifluoromethyl)phenyl]-5-(4-ethylpiperazin-1-yl)-3,6-dihydro-1H-triazolo[4,5-d]pyrimidin-7-one (300 mg, 688.9 mol) in DMF (20.0 mL) was added [acetoxy(phenyl)-iodanyl]acetate (310 mg, 964 mol). The mixture was stirred at 20° C. for 12 hrs. LC-MS showed the reaction was complete. The reaction mixture was diluted with H2O (20.0 mL) and extracted with CH2Cl2 (20.0 mL×2). The combined organic layers were washed with brine (20.0 mL), dried over Na2SO4, filtered and concentrated under reduced pressure to give a residue. The residue was purified by prep-HPLC (column: Welch Xtimate C18 40*200 mm 7 um; mobile phase: [water(NH3H2O+NH4HCO3)-ACN]; gradient: 12%-52% B over 25 min) to give 2-[4-cyclopropyl-2-(trifluoromethyl)phenyl]-5-(4-ethylpiperazin-1-yl)-6H-triazolo[4,5-d]pyrimidin-7-one (83.9 mg, 193 mol, 28.0% yield, 99.7% purity) as a white solid.

LCMS: m/z=434.1 (M+H)+, Rt=1.181 min

1H NMR: (400 MHz, CD3OD)

δ 7.65-7.71 (m, 2H), 7.53 (dd, J=8.4, 1.7 Hz, 1H), 3.58-3.70 (m, 5H), 2.37-2.43 (m, 4H), 2.32-2.37 (m, 2H), 2.15-2.23 (m, 1H), 1.07-1.13 (m, 2H), 1.03 (t, J=7.2 Hz, 3H), 0.85-0.90 (m, 2H)

### Example 101: Preparation of 2-(4-cyclopropyl-2,6-dimethylphenyl)-5-((2-(dimethylamino)ethyl)(methyl)amino)-2,6-dihydro-7H-[1,2,3]triazolo[4,5-d]pyrimidin-7-one (Compound 101)

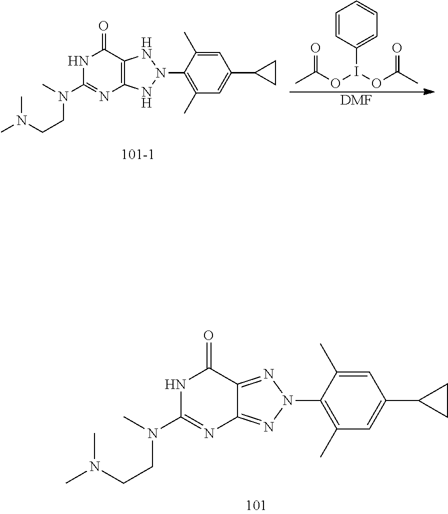

To a solution of 2-(4-cyclopropyl-2,6-dimethylphenyl)-5-((2-(dimethylamino)ethyl)(methyl)amino)-1,2,3,6-tetrahydro-7H-[1,2,3]triazolo[4,5-d]pyrimidin-7-one (500 mg, 1.30 mmol) in DMF (5 mL) was added phenyl-l3-iodanediyl diacetate (588 mg, 1.83 mmol). The mixture was stirred at 20° C. for 16 hrs. LCMS showed reaction was complete. The reaction mixture was quenched by addition NaHCO3 (5 mL) at 25° C., and then diluted with H2O 10 mL and extracted with CH2Cl2 60 mL (30 mL×2). The combined organic layers were dried over Na2SO4, filtered and concentrated under reduced pressure to give a residue, which was purified by prep-HPLC (column: Welch Xtimate C18 40*200 mm 7 um; mobile phase: [water (NH3H2O+NH4HCO3)—ACN]; gradient: 16%-56% B over 25 min) to give 2-(4-cyclopropyl-2,6-dimethylphenyl)-5-((2-(dimethylamino)ethyl)(methyl)amino)-2,6-dihydro-7H-[1,2,3]triazolo[4,5-d]pyrimidin-7-one (121 mg, 24.3% yield, 99.5% purity) as a yellow oil.

LCMS: m/z=383.2 (M+1+H)+, Rt=4.428 min

1H NMR: (400 MHz, MeOD)

δ 6.93 (s, 2H), 3.66-3.69 (m, 2H), 3.19 (s, 3H), 2.91-2.95 (m, 2H), 2.60 (s, 6H), 1.96 (s, 6H), 1.90-1.96 (m, 1H), 0.99-1.05 (m, 2H) 0.73-0.77 (m, 2H)

### Example 102: Preparation of 2-(4-cyclopropyl-2,6-dimethylphenyl)-5-(4-ethylpiperazin-1-yl)-2,6-dihydro-7H-[1,2,3]triazolo[4,5-d]pyrimidin-7-one (Compound 102)

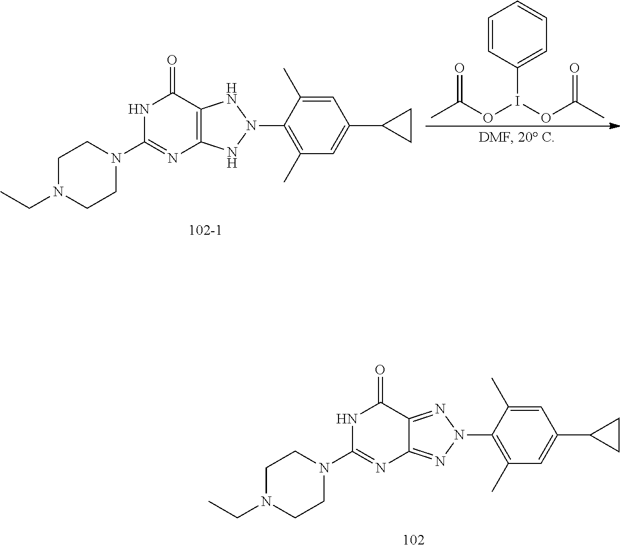

To a solution of 2-(4-cyclopropyl-2,6-dimethylphenyl)-5-(4-ethylpiperazin-1-yl)-1,2,3,6-tetrahydro-7H-[1,2,3]triazolo[4,5-d]pyrimidin-7-one (300 mg, 758.5 mol) in DMF (20.0 mL) was added [acetoxy(phenyl)-iodanyl]acetate (342 mg, 1.06 mmol). The mixture was stirred at 20° C. for 12 hrs. LCMS showed the reaction was complete. The reaction mixture was diluted with H2O (20.0 mL) and extracted with CH2Cl2 (20.0 mL×2). The combined organic layers were washed with brine (20.0 mL), dried over Na2SO4, filtered and concentrated under reduced pressure to give a residue. The residue was purified by prep-HPLC to give 2-(4-cyclopropyl-2,6-dimethylphenyl)-5-(4-ethylpiperazin-1-yl)-2,6-dihydro-7H-[1,2,3]triazolo[4,5-d]pyrimidin-7-one was obtained (50.18 mg, 16.7% yield, 99.4% purity) as a yellow solid.

LCMS: m/z=394.1 (M+H)+, Rt=1.166 min

1H NMR: (400 MHz, CD3OD)

δ 7.58-7.71 (m, 1H), 6.84 (s, 2H), 4.04 (s, 1H), 3.81 (s, 4H), 2.36-2.43 (m, 6H), 2.29-2.36 (m, 7H), 1.82-1.89 (m, 1H), 1.03 (t, J=7.2 Hz, 3H), 0.90-0.96 (m, 2H), 0.63-0.70 (m, 2H)

### Example 103: Preparation of 2-(2-chloro-4-cyclopropyl-6-fluorophenyl)-5-morpholino-2,6-dihydro-7H-[1,2,3]triazolo[4,5-d]pyrimidin-7-one (Compound 103)

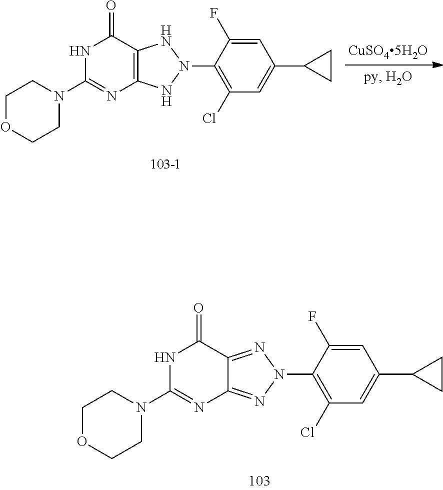 [SMILES: O=c1[nH]c(N2CCOCC2)nc2c1NN(c1c(F)cc(C3CC3)cc1Cl)N2 ; O=c1[nH]c(N2CCOCC2)nc2nn(-c3c(F)cc(C4CC4)cc3Cl)nc12]

To a solution of 2-(2-chloro-4-cyclopropyl-6-fluoro-phenyl)-5-morpholino-3,6-dihydro-1H-triazolo[4,5-d]pyrimidin-7-one (200 mg, 509 mol) in H2O (3 mL) and Py (3 mL) was added CuSO4·5H2O (381 mg, 1.53 mmol). The mixture was stirred at 100° C. for 8 hrs. LC-MS showed the reaction was completed. The reaction mixture was diluted with H2O (10 mL) and extracted with EtOAc (10 mL×2). The combined organic layers were dried over Na2SO4, filtered and concentrated under reduced pressure to give a residue. The residue was purified by prep-HPLC (column: Welch Xtimate C18 40*200 mm 7 um; mobile phase: [water(HCl)-ACN]; gradient: 18%-58% B over 20.5 min) to give 2-(2-chloro-4-cyclopropyl-6-fluoro-phenyl)-5-morpholino-6H-triazolo[4,5-d]pyrimidin-7-one (60.6 mg, 30.4% yield, 100% purity) as a white solid.

LCMS: m/z=319.0 (M+H)+, Rt=0.608 min

1H NMR: (400 MHz, DMSO-d6)

δ 11.55 (s, 1H), 7.42 (s, 1H), 7.32 (dd, J=10.8, 1.6 Hz, 1H), 3.65-3.69 (m, 4H), 3.60-3.65 (m, 4H), 2.07-2.15 (m, 1H), 1.06-1.15 (m, 2H), 0.88-0.95 (m, 2H)

### Example 104: Preparation of 2-(4-cyclopropyl-2-fluoro-6-methylphenyl)-5-morpholino-2,6-dihydro-7H-[1,2,3]triazolo[4,5-d]pyrimidin-7-one (Compound 104)

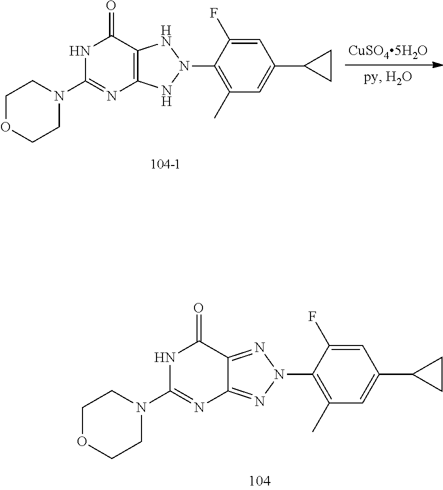 [SMILES: Cc1cc(C2CC2)cc(F)c1N1Nc2nc(N3CCOCC3)[nH]c(=O)c2N1 ; Cc1cc(C2CC2)cc(F)c1-n1nc2nc(N3CCOCC3)[nH]c(=O)c2n1]

To a solution of 2-(4-cyclopropyl-2-fluoro-6-methyl-phenyl)-5-morpholino-3,6-dihydro-1H-triazolo[4,5-d]pyrimidin-7-one (200 mg, 537 mol) in H2O (3 mL) and Py (3 mL) was added CuSO4·5H2O (402 mg, 1.61 mmol). The mixture was stirred at 100 20° C. for 8 hrs. The mixture was stirred at 100° C. for 8 hrs. LC-MS showed the reaction was completed. The reaction mixture was diluted with H2O (10 mL) and extracted with EtOAc (10 mL×2). The combined organic layers were dried over Na2SO4, filtered and concentrated under reduced pressure to give a residue. The residue was purified by prep-HPLC (column: Welch Xtimate C18 40*200 mm 7 um; mobile phase: [water(HCl)-ACN]; gradient: 20%-60% B over 20.5 min) to give 2-(4-cyclopropyl-2-fluoro-6-methyl-phenyl)-5-morpholino-6H-triazolo[4,5-d]pyrimidin-7-one (69.12 mg, 34.7% yield, 100% purity) as a white solid.

LCMS: m/z=371.0 (M+H)+, Rt=0.618 min

1H NMR: (400 MHz, DMSO-d6)

δ 11.51 (s, 1H), 7.00-7.14 (m, 2H), 3.65-3.70 (m, 4H), 3.57-3.64 (m, 4H), 1.98-2.09 (m, 4H), 1.00-1.11 (m, 2H), 0.78-0.87 (m, 2H)

### Example 105: Preparation of 2-(4-cyclopropyl-2-fluoro-6-(trifluoromethyl)phenyl)-5-morpholino-2,6-dihydro-7H-[1,2,3]triazolo[4,5-d]pyrimidin-7-one (Compound 105)

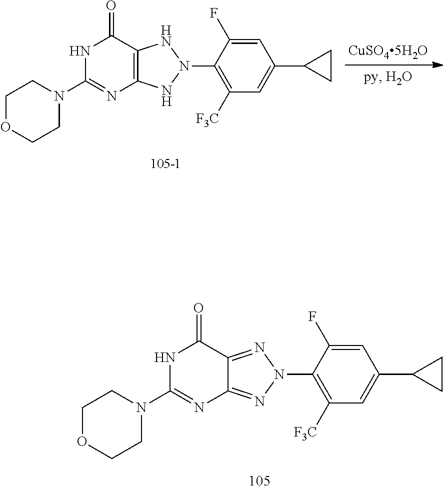 [SMILES: O=c1[nH]c(N2CCOCC2)nc2c1NN(c1c(F)cc(C3CC3)cc1C(F)(F)F)N2 ; O=c1[nH]c(N2CCOCC2)nc2nn(-c3c(F)cc(C4CC4)cc3C(F)(F)F)nc12]

To a solution of 2-[4-cyclopropyl-2-fluoro-6-(trifluoromethyl)phenyl]-5-morpholino-3,6-dihydro-1H-triazolo[4,5-d]pyrimidin-7-one (300 mg, 704 mol) in H2O (5 mL) and Py (5 mL) was added CuSO4·5H2O (527 mg, 2.11 mmol). The mixture was stirred at 100° C. for 8 hrs. LC-MS showed the reaction was completed. The reaction mixture was diluted with H2O (10 mL) and extracted with EtOAc (10 mL×2). The combined organic layers were dried over Na2SO4, filtered and concentrated under reduced pressure to give a residue. The residue was purified by prep-HPLC (column: Welch Xtimate C18 40*200 mm 7 um; mobile phase: [water(HCl)-ACN]; gradient: 20%-60% B over 20.5 min) to give 2-[4-cyclopropyl-2-fluoro-6-(trifluoromethyl)phenyl]-5-morpholino-6H-triazolo[4,5-d]pyrimidin-7-one (73.48 mg, 24.2% yield, 98.55% purity) as a yellow solid.

LCMS: m/z=425.0 (M+H)+, Rt=0.626 min

1H NMR: (400 MHz, DMSO-d6)

δ 11.56 (s, 1H), 7.67 (s, 1H), 7.62 (d, J=10.8 Hz, 1H), 3.65-3.70 (m, 4H), 3.60-3.65 (m, 4H), 2.19-2.29 (m, 1H), 1.12-1.19 (m, 2H), 0.94-1.00 (m, 2H)

### Example 106: Preparation of 2-(4-cyclopropyl-2-fluoro-6-methylphenyl)-5-(4-ethylpiperazin-1-yl)-2,6-dihydro-7H-[1,2,3]triazolo[4,5-d]pyrimidin-7-one (Compound 106)

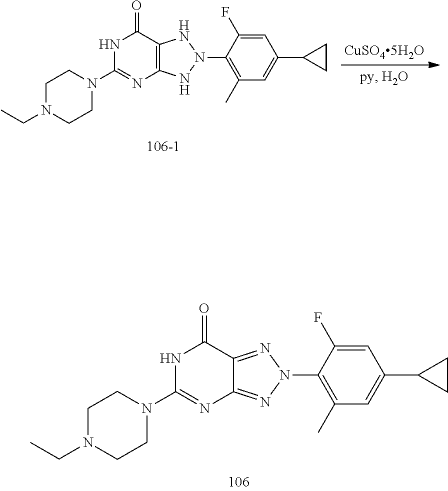 [SMILES: CCN1CCN(c2nc3c(c(=O)[nH]2)NN(c2c(C)cc(C4CC4)cc2F)N3)CC1 ; CCN1CCN(c2nc3nn(-c4c(C)cc(C5CC5)cc4F)nc3c(=O)[nH]2)CC1]

To a solution of 2-(4-cyclopropyl-2-fluoro-6-methyl-phenyl)-5-(4-ethylpiperazin-1-yl)-3,6-dihydro-1H-triazolo[4,5-d]pyrimidin-7-one (300 mg, 751 mol) in H2O (5 mL) and Py (5 mL) was added copper; sulfate; pentahydrate (562 mg, 2.25 mmol). The mixture was stirred at 100° C. for 8 hrs. TLC (Petroleum ether/EtOAc=1/1) showed the reaction was completed. The reaction mixture was diluted with EtOAc (20 mL) and washed with H2O (20 mL×2), dried over Na2SO4, filtered and concentrated under reduced pressure to give a residue. The residue was purified by column chromatography (SiO2, Petroleum ether/EtOAc=1/0 to 1/1) to 2-(4-cyclopropyl-2-fluoro-6-methyl-phenyl)-5-(4-ethylpiperazin-1-yl)-6H-triazolo[4,5-d]pyrimidin-7-one (59.15 mg, 19.9% yield, 99.36% purity) as a gray solid.

1H NMR: (400 MHz, DMSO-d6)

δ 11.20-11.63 (m, 1H), 6.99-7.13 (m, 2H), 3.54-3.69 (m, 4H), 2.40-2.48 (m, 4H), 2.36 (q, J=7.2 Hz, 2H), 1.98-2.08 (m, 4H), 0.98-1.10 (m, 5H), 0.79-0.86 (m, 2H)

LCMS: m/z=398.2 (M+H)+, Rt=1.545 min

### Example 107: Preparation of (R)-2-(4-cyclopropyl-2,6-dimethylphenyl)-5-(3-methylmorpholino)-2H-[1,2,3]triazolo[4,5-d]pyrimidin-7(6H)-one (Compound 107)

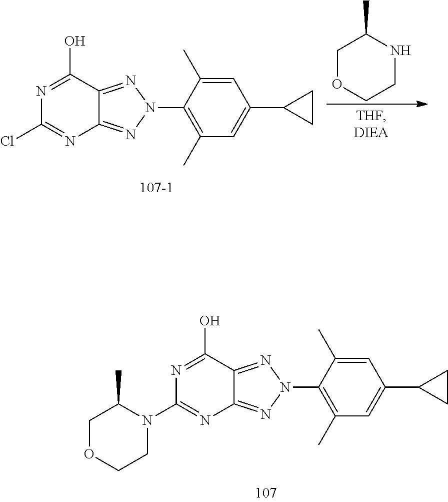

To a solution of 5-chloro-2-(4-cyclopropyl-2,6-dimethyl-phenyl)triazolo[4,5-d]pyrimidin-7-ol (200 mg, 633 mol) and (3R)-3-methylmorpholine (640 mg, 6.33 mmol) in THF (10 mL) was added DIEA (818 mg, 6.33 mmol, 1.10 mL). The mixture was stirred at 80° C. for 12 hrs. TLC (Petroleum ether/EtOAc=1/1, Rf=0.2) showed the reaction was completed. The reaction mixture was diluted with water (30 mL) and extracted with EtOAc (30 mL×3). The combined organic layers were dried over Na2SO4, filtered and the filtrate was concentrated to yield a residue, which was purified by column chromatography (SiO2, Petroleum ether/EtOAc=100/1 to 1/1) to give (R)-2-(4-cyclopropyl-2,6-dimethylphenyl)-5-(3-methylmorpholino)-2H-[1,2,3]triazolo[4,5-d]pyrimidin-7(6H)-one (104 mg, 264 mmol, 41.7% yield, 96.0% purity) as a yellow solid.

1H NMR: (400 MHz, DMSO-d6)

δ 11.35 (s, 1H), 6.98 (s, 2H), 4.41-4.46 (m, 1H), 3.83-4.03 (m, 2H), 3.65-3.74 (m, 1H), 3.56-3.64 (m, 1H), 3.41-3.49 (m, 1H), 3.19-3.24 (m, 1H), 1.93-1.98 (m, 1H), 1.91 (s, 6H), 1.24 (d, J=6.4 Hz, 3H), 0.96-1.03 (m, 2H), 0.73-0.79 (m, 2H)

LCMS: m/z=381.0 (M+H)+, Rt=2.334 min

### Example 108 and 109: Preparation of 2-(4-cyclopropyl-2,6-dimethyl-phenyl)-6-methyl-5-morpholino-triazolo[4,5-d]pyrimidin-7-one and 4-[2-(4-cyclopropyl-2,6-dimethyl-phenyl)-7-methoxy-triazolo[4,5-d]pyrimidin-5-yl]morpholine (Compound 108 and 109)

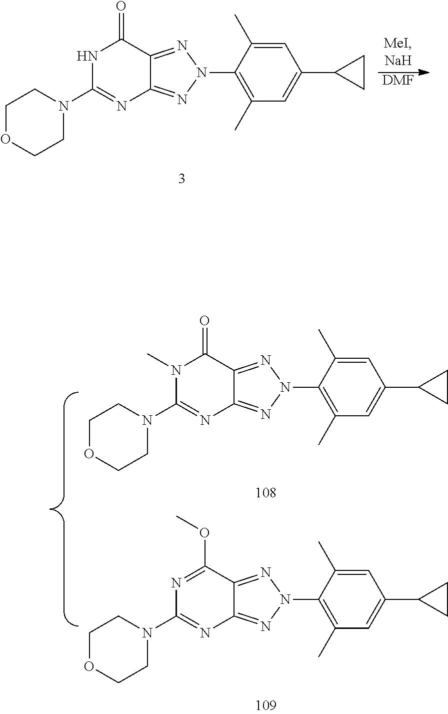

To a solution of 2-(4-cyclopropyl-2,6-dimethyl-phenyl)-5-morpholino-6H-triazolo[4,5-d]pyrimidin-7-one (932 mg, 2.54 mmol) in DMF (15 mL) was added NaH (152 mg, 3.82 mmol, 60% purity) at 0° C. The mixture was stirred at 0° C. for 2 hrs, then Mel (1.08 g, 7.63 mmol, 475 L) was added. After addition, the mixture was stirred at 25° C. for 2 hrs. LC-MS showed the reaction was completed. The reaction mixture was diluted with water (100 mL) and extracted with EtOAc (100 mL×3). The combined organic layers were dried over Na2SO4, filtered and the filtrate was concentrated to yield a residue, which was purified by prep-HPLC (column: F-Prepulite XP tC 18 40*200 mm*7 um; mobile phase: [water(NH4HCO3)-ACN]; gradient: 42%-82% B over 14 min) to give 4-[2-(4-cyclopropyl-2,6-dimethyl-phenyl)-7-methoxy-triazolo[4,5-d]pyrimidin-5-yl]morpholine (199 mg, 20.4% yield, 100% purity) as a white solid and 2-(4-cyclopropyl-2,6-dimethyl-phenyl)-6-methyl-5-morpholino-triazolo[4,5-d]pyrimidin-7-one (132 mg, 340 mol, 13.3% yield, 100% purity) as a white solid.

LCMS: m/z=381.3 (M+H)+, Rt=6.483 min & Rt=5.600 min

1H NMR: (400 MHz, DMSO-d6)

δ 6.99 (s, 2H), 4.12 (s, 3H), 3.75-3.83 (m, 4H), 3.63-3.75 (m, 4H), 1.92-1.99 (m, 1H), 1.88 (s, 6H), 0.92-1.08 (m, 2H), 0.70-0.81 (m, 2H)

LCMS: m/z=381.4 (M+H)+, Rt=2.898 min

1H NMR: (400 MHz, DMSO-d6)

δ 6.99 (s, 2H), 3.74-3.79 (m, 4H), 3.51 (s, 3H), 3.20-3.24 (m, 4H), 1.92-2.00 (m, 1H), 1.90 (s, 6H), 0.95-1.06 (m, 2H), 0.71-0.82 (m, 2H)

LCMS: m/z=381.4 (M+H)+, Rt=2.459 min

### Examples 55-111

Compounds 55-111 can be prepared following one of the procedures described above in Examples 51-54 using commercially available starting materials and agents.

### Example 112: Evaluation of NLRP3 Inhibitors

Compounds 1 and 2 were found to inhibit NLRP3 activation to a degree indicated by its IC₅₀ value shown in Table-2 and tested with the following means.

### Cell Culture and Inflammasome Activation Assays

THP-1 cell lines (procell, #CL-0233) were cultured in RPMI-1640 medium (Gibco, #C11875500BT) supplemented with 10% FBS, 100 U/ml penicillin, 100 g/ml streptomycin and 0.05 mM β-mercaptoethanol (BME). THP-1 cells were seeded at 5×10⁵/ml in 12 well plates and were differentiated into macrophages by incubation with 5 ng/ml PMA (Sigma, #P8139-1MG) for 48 h. Cells were washed with PBS and then were primed with 50 ng/ml LPS (InvivoGen, #tlrl-b5lps) for 4 h. Medium were removed and replaced with serum free medium (SFM) containing compound (2.5-40 nM) or MCC950 (5-25 nM) for 30 min. Cells were then stimulated with 15 μM nigericin (Invivogen, #tlrl-nig) for 45 min.

### Enzyme-Linked Immunosorbent Assay (ELISA)

The culture supernatant of THP-1 was removed and analyzed using Human IL-1β ELISA Kit according to the manufacturer's instructions (Beyotime, #PI305). The IL-1β concentration was measured at 450 nm wavelength of absorbance and calculated.

### Western Blotting

The protein of supernatants was concentrated using StrataClean™ resin (Agilent, #400714) according to the manufacturer's instructions. The protein samples were resolved on 4-20% SDS-PAGE gels and transferred onto 0.45 PVDF membrane (Immobilon-P #IPVH00010) using a wet transfer system. Membranes were blocked in 5% (w/v) dried milk in PBST for 1 h at room temperature. Membranes were incubated with primary antibody (Adipogen, #AG-20B-0048B-C100) diluted in Primary Antibody Dilution Buffer (Beyotime, #P0023A-100 ml) according to the manufacturer's instructions, followed by incubation with the IgG(H+L) (HRP-labeled Goat Anti-Mouse IgG(H+L) (Beyotime, #A0216) diluted in 1% (w/v) dried milk in PBST for 1 h. The protein expression was visualized using Super ECL Detection Reagent (YEASEN,

TABLE 2
NLRP3 inhibitory activity

| | | THP-1 | |
|---|---|---|---|
| | | pyroptosis | IL-1β |
| | | assay IC₅₀ | Assay |
| Example | | (Caspase-1) | IC₅₀ |
| No. | Structure | (nM) | (nM) |
| 1 | 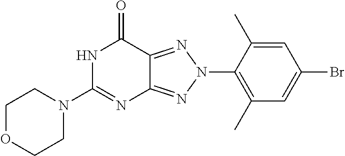 [SMILES: Cc1cc(Br)cc(C)c1-n1nc2nc(N3CCOCC3)[nH]c(=O)c2n1] | <2.5 | <20 |
| 2 | 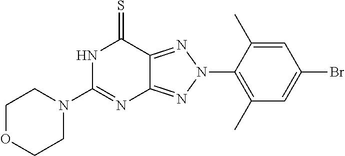 [SMILES: Cc1cc(Br)cc(C)c1-n1nc2nc(N3CCOCC3)[nH]c(=S)c2n1] | <1600 | <1600 |
| 3 | 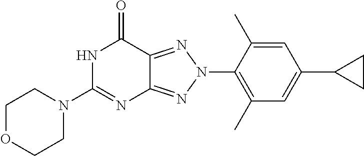 [SMILES: Cc1cc(C2CC2)cc(C)c1-n1nc2nc(N3CCOCC3)[nH]c(=O)c2n1] | <5 | <15 |
| 4 | 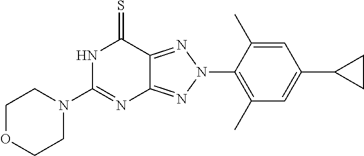 [SMILES: Cc1cc(C2CC2)cc(C)c1-n1nc2nc(N3CCOCC3)[nH]c(=S)c2n1] | >1000 | >1000 |
| 27 | 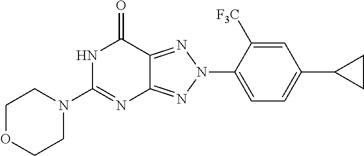 [SMILES: O=c1[nH]c(N2CCOCC2)nc2nn(-c3ccc(C4CC4)cc3C(F)(F)F)nc12] | — | — |
| 51 | 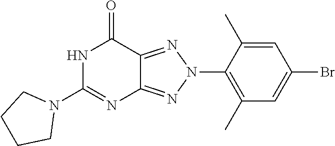 [SMILES: Cc1cc(Br)cc(C)c1-n1nc2nc(N3CCCC3)[nH]c(=O)c2n1] | <5 | <5 |
| 52 | 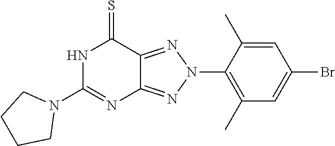 [SMILES: Cc1cc(Br)cc(C)c1-n1nc2nc(N3CCCC3)[nH]c(=S)c2n1] | <50 | <500 |
| 53 | 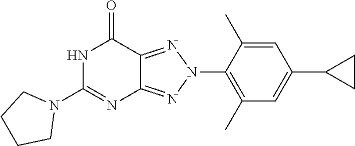 [SMILES: Cc1cc(C2CC2)cc(C)c1-n1nc2nc(N3CCCC3)[nH]c(=O)c2n1] | <1 | <5 |
| 54 | 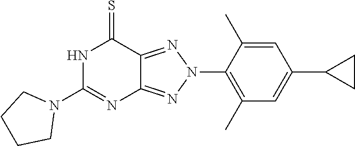 [SMILES: Cc1cc(C2CC2)cc(C)c1-n1nc2nc(N3CCCC3)[nH]c(=S)c2n1] | <100 | <100 |
| 61 | 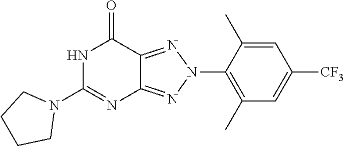 [SMILES: Cc1cc(C(F)(F)F)cc(C)c1-n1nc2nc(N3CCCC3)[nH]c(=O)c2n1] | — | — |
| 63 | 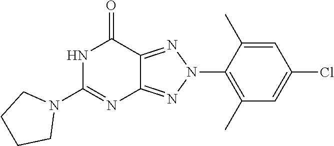 [SMILES: Cc1cc(Cl)cc(C)c1-n1nc2nc(N3CCCC3)[nH]c(=O)c2n1] | 10 | — |
| 75 | 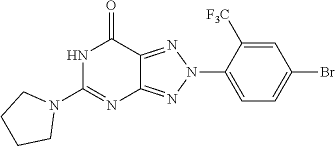 [SMILES: O=c1[nH]c(N2CCCC2)nc2nn(-c3ccc(Br)cc3C(F)(F)F)nc12] | 2.5 | <10 |
| 77 | 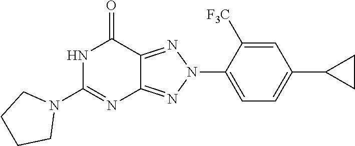 [SMILES: O=c1[nH]c(N2CCCC2)nc2nn(-c3ccc(C4CC4)cc3C(F)(F)F)nc12] | <10 | <10 |
| 91 | 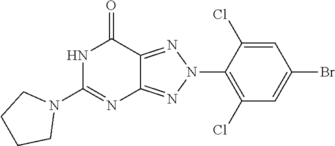 [SMILES: O=c1[nH]c(N2CCCC2)nc2nn(-c3c(Cl)cc(Br)cc3Cl)nc12] | 7.5 | <15 |
| 95 | 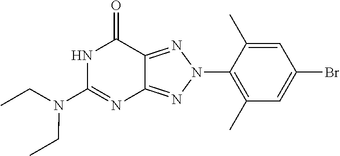 [SMILES: CCN(CC)c1nc2nn(-c3c(C)cc(Br)cc3C)nc2c(=O)[nH]1] | <6.2 | <6.2 |
| 96 | 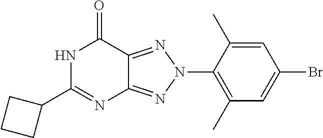 [SMILES: Cc1cc(Br)cc(C)c1-n1nc2nc(C3CCC3)[nH]c(=O)c2n1] | — | — |
| 97 | 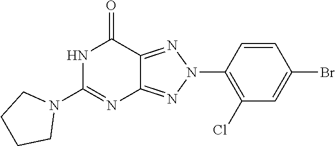 [SMILES: O=c1[nH]c(N2CCCC2)nc2nn(-c3ccc(Br)cc3Cl)nc12] | 5 | <20 |
| 98 | 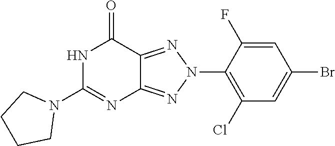 [SMILES: O=c1[nH]c(N2CCCC2)nc2nn(-c3c(F)cc(Br)cc3Cl)nc12] | 7.5 | <15 |
| 99 | 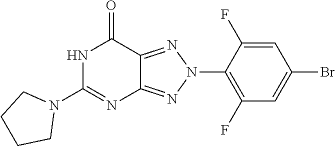 [SMILES: O=c1[nH]c(N2CCCC2)nc2nn(-c3c(F)cc(Br)cc3F)nc12] | <10 | <20 |
| 100 | 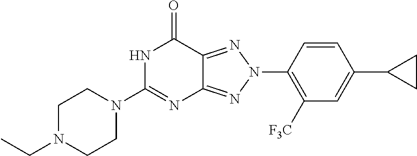 [SMILES: CCN1CCN(c2nc3nn(-c4ccc(C5CC5)cc4C(F)(F)F)nc3c(=O)[nH]2)CC1] | <100 | <200 |
| 101 | 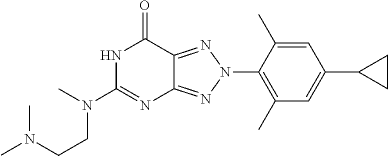 [SMILES: Cc1cc(C2CC2)cc(C)c1-n1nc2nc(N(C)CCN(C)C)[nH]c(=O)c2n1] | <10 | <100 |
| 102 | 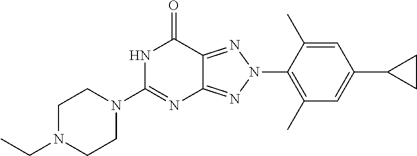 [SMILES: CCN1CCN(c2nc3nn(-c4c(C)cc(C5CC5)cc4C)nc3c(=O)[nH]2)CC1] | <40 | >100 |
| 103 | 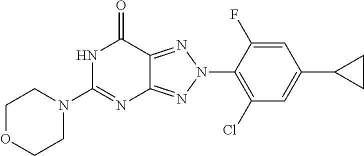 [SMILES: O=c1[nH]c(N2CCOCC2)nc2nn(-c3c(F)cc(C4CC4)cc3Cl)nc12] | <10 | <10 |
| 104 | 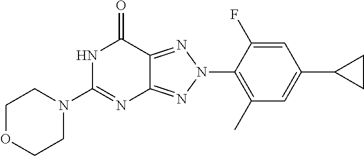 [SMILES: Cc1cc(C2CC2)cc(F)c1-n1nc2nc(N3CCOCC3)[nH]c(=O)c2n1] | <6 | <10 |
| 105 | 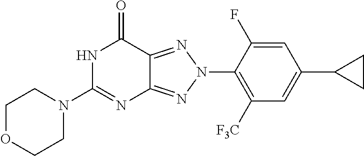 [SMILES: O=c1[nH]c(N2CCOCC2)nc2nn(-c3c(F)cc(C4CC4)cc3C(F)(F)F)nc12] | <6 | <10 |
| 106 | 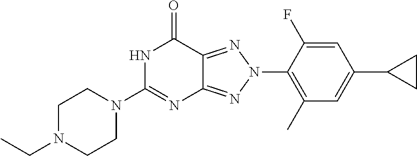 [SMILES: CCN1CCN(c2nc3nn(-c4c(C)cc(C5CC5)cc4F)nc3c(=O)[nH]2)CC1] | >100 | >100 |
| 107 | 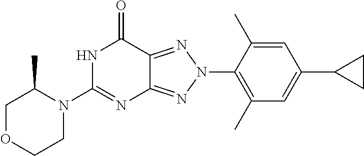 [SMILES: Cc1cc(C2CC2)cc(C)c1-n1nc2nc(N3CCOC[C@H]3C)[nH]c(=O)c2n1] | — | <33 |
| 108 | 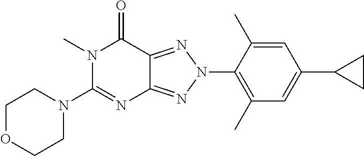 [SMILES: Cc1cc(C2CC2)cc(C)c1-n1nc2nc(N3CCOCC3)n(C)c(=O)c2n1] | — | <500 |
| 109 | 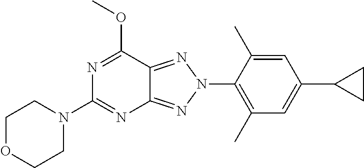 [SMILES: COc1nc(N2CCOCC2)nc2nn(-c3c(C)cc(C4CC4)cc3C)nc12] | — | >10000 |
| 110 | 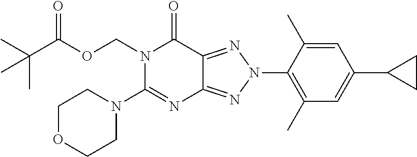 [SMILES: Cc1cc(C2CC2)cc(C)c1-n1nc2nc(N3CCOCC3)n(COC(=O)C(C)(C)C)c(=O)c2n1] | — | — |
| 111 | 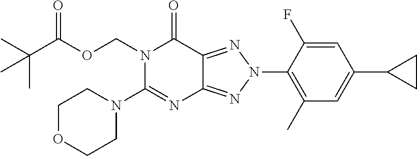 [SMILES: Cc1cc(C2CC2)cc(F)c1-n1nc2nc(N3CCOCC3)n(COC(=O)C(C)(C)C)c(=O)c2n1] | — | — |

### Example 113: ADMET Properties of the Above Exemplary NLRP3 Inhibitors Predicted with Machine-Learning Models

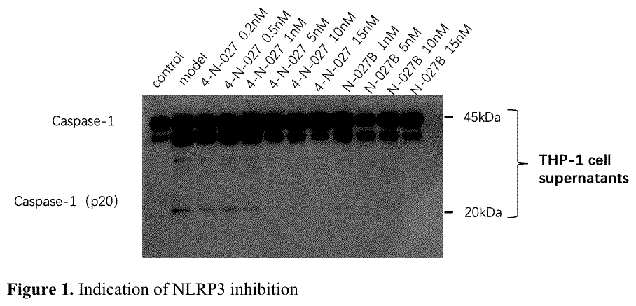

All compounds in Table 1 were systematically studied for their drug-related properties including absorption, distribution, metabolism, and toxify with various machine learning models. The results from ADMETLab2.0 models are listed in Table 3 and the outcome distributions are plotted in FIG. 1.

FIG. 1. Results of Western blots for THP-1 cell-based pyroptosis assay using the cleavage of caspase-1 into p20 as reporting indication of NLRP3 inhibition. Reduction or absence of p20 indicates partially or fully inhibited activation of NLRP3. Molecule N-027 (Compound #51) and N-027B (Compound #53) inhibits NLRP3 inflammasome activation in macrophages, N-027 IC₅₀ (caspase-1)<5 nM; N-027B IC₅₀ (caspase-1) 1 nM. THP-1 cells were primed for 4 h with LPS (50 ng/ml), and then LPS-primed THP-1 cells were incubated with N-027 or N-027B for 30 min and then stimulated with nigericin (15 μM) for 45 min. Caspase-1 (p20) in supernatants were measured by Western blots.

The properties predicted and their design empirical decision rules are as follows. Lipinski Rule: <2 violations: excellent (green); ≥2 violations: poor (red). Drug-Likeness: >0.67: excellent (green); ≤0.67: poor (red). Synthetic accessibility Score: ≤6: excellent (green); >6: poor (red). Half-Life: 0-0.3: excellent (green); 0.3-0.7: medium (yellow); 0.7-1.0(++): poor (red). Human Oral Bioavailability 30%: 0-0.3: excellent (green); 0.3-0.7: medium (yellow); 0.7-1.0(++): poor (red). # of Pan Assay Interference Compounds: 0: excellent (green). Chance of having Rat Oral Acute Toxicity: 0-0.3: excellent (green); 0.3-0.7: medium (yellow); 0.7-1.0(++): poor (red). Chance of having significant Blood-Brain Barrier: 0-0.3: excellent (green); 0.3-0.7: medium (yellow); 0.7-1.0(++): poor (red).

TABLE 3

| No. | MW | Lipinski | QED | Synth | T12 | F(30%) | PAINS | ROA | BBB | Pfizer |
|---|---|---|---|---|---|---|---|---|---|---|
| 1 | 404.06 | Accepted | 0.699 | 3.042 | 0.562 | 0.552 | 0 | 0.183 | 0.03 | Accepted |
| 2 | 420.04 | Accepted | 0.642 | 3.223 | 0.627 | 0.171 | 0 | 0.179 | 0.036 | Rejected |
| 3 | 366.18 | Accepted | 0.761 | 3.067 | 0.315 | 0.074 | 0 | 0.629 | 0.034 | Accepted |
| 4 | 382.16 | Accepted | 0.702 | 3.234 | 0.326 | 0.007 | 0 | 0.668 | 0.037 | Rejected |
| 5 | 340.16 | Accepted | 0.756 | 2.951 | 0.642 | 0.143 | 0 | 0.217 | 0.018 | Accepted |
| 6 | 356.14 | Accepted | 0.712 | 3.132 | 0.71 | 0.069 | 0 | 0.28 | 0.022 | Rejected |
| 7 | 351.14 | Accepted | 0.727 | 3.115 | 0.704 | 0.612 | 0 | 0.513 | 0.017 | Accepted |
| 8 | 367.12 | Accepted | 0.694 | 3.288 | 0.755 | 0.18 | 0 | 0.402 | 0.014 | Accepted |
| 9 | 344.14 | Accepted | 0.749 | 3.009 | 0.458 | 0.354 | 0 | 0.504 | 0.023 | Accepted |
| 10 | 360.12 | Accepted | 0.708 | 3.19 | 0.533 | 0.034 | 0 | 0.376 | 0.026 | Accepted |
| 11 | 394.14 | Accepted | 0.715 | 3.073 | 0.328 | 0.036 | 0 | 0.459 | 0.025 | Accepted |
| 12 | 410.11 | Accepted | 0.654 | 3.237 | 0.34 | 0.014 | 0 | 0.516 | 0.027 | Rejected |
| 13 | 360.11 | Accepted | 0.747 | 3.003 | 0.478 | 0.026 | 0 | 0.201 | 0.017 | Accepted |
| 14 | 376.09 | Accepted | 0.693 | 3.184 | 0.557 | 0.009 | 0 | 0.204 | 0.013 | Rejected |
| 15 | 376.15 | Accepted | 0.751 | 3.177 | 0.38 | 0.523 | 0 | 0.038 | 0.016 | Accepted |
| 16 | 392.12 | Accepted | 0.69 | 3.346 | 0.394 | 0.094 | 0 | 0.055 | 0.017 | Rejected |
| 17 | 390.16 | Accepted | 0.737 | 3.199 | 0.329 | 0.043 | 0 | 0.145 | 0.024 | Accepted |
| 18 | 406.14 | Accepted | 0.672 | 3.364 | 0.341 | 0.022 | 0 | 0.189 | 0.02 | Rejected |
| 19 | 368.2 | Accepted | 0.761 | 3.032 | 0.425 | 0.075 | 0 | 0.117 | 0.033 | Accepted |
| 20 | 384.17 | Accepted | 0.698 | 3.201 | 0.466 | 0.099 | 0 | 0.153 | 0.032 | Rejected |
| 21 | 410.13 | Accepted | 0.704 | 3.112 | 0.401 | 0.181 | 0 | 0.894 | 0.028 | Accepted |
| 22 | 426.11 | Accepted | 0.644 | 3.271 | 0.457 | 0.095 | 0 | 0.861 | 0.04 | Accepted |
| 23 | 392.14 | Accepted | 0.719 | 3.11 | 0.41 | 0.543 | 0 | 0.41 | 0.019 | Accepted |
| 24 | 408.12 | Accepted | 0.665 | 3.272 | 0.48 | 0.114 | 0 | 0.293 | 0.024 | Accepted |
| 25 | 444.02 | Accepted | 0.651 | 3.013 | 0.259 | 0.077 | 0 | 0.762 | 0.062 | Accepted |
| 26 | 459.99 | Accepted | 0.59 | 3.182 | 0.245 | 0.02 | 0 | 0.814 | 0.046 | Rejected |
| 27 | 406.14 | Accepted | 0.718 | 3.039 | 0.119 | 0.019 | 0 | 0.928 | 0.042 | Accepted |
| 28 | 422.11 | Accepted | 0.651 | 3.195 | 0.112 | 0.003 | 0 | 0.944 | 0.032 | Rejected |
| 29 | 408.15 | Accepted | 0.716 | 3.014 | 0.192 | 0.024 | 0 | 0.738 | 0.035 | Accepted |
| 30 | 424.13 | Accepted | 0.644 | 3.172 | 0.176 | 0.014 | 0 | 0.74 | 0.028 | Rejected |
| 31 | 400.07 | Accepted | 0.708 | 2.967 | 0.25 | 0.007 | 0 | 0.562 | 0.028 | Accepted |
| 32 | 416.04 | Accepted | 0.646 | 3.135 | 0.243 | 0.004 | 0 | 0.596 | 0.013 | Rejected |
| 33 | 434.09 | Accepted | 0.624 | 3.039 | 0.137 | 0.016 | 0 | 0.877 | 0.158 | Accepted |
| 34 | 450.07 | Accepted | 0.474 | 3.194 | 0.125 | 0.006 | 0 | 0.931 | 0.044 | Rejected |
| 35 | 384.1 | Accepted | 0.674 | 2.979 | 0.22 | 0.031 | 0 | 0.678 | 0.035 | Accepted |
| 36 | 400.07 | Accepted | 0.527 | 3.148 | 0.21 | 0.009 | 0 | 0.752 | 0.025 | Accepted |
| 37 | 432.1 | Accepted | 0.63 | 3.09 | 0.213 | 0.116 | 0 | 0.713 | 0.092 | Accepted |
| 38 | 448.07 | Accepted | 0.484 | 3.243 | 0.207 | 0.024 | 0 | 0.793 | 0.042 | Accepted |
| 39 | 450.09 | Accepted | 0.612 | 3.097 | 0.223 | 0.059 | 0 | 0.945 | 0.346 | Accepted |
| 40 | 466.06 | Accepted | 0.467 | 3.246 | 0.199 | 0.024 | 0 | 0.963 | 0.092 | Accepted |
| 41 | 443.95 | Accepted | 0.651 | 3.055 | 0.434 | 0.012 | 0 | 0.378 | 0.063 | Accepted |
| 42 | 459.93 | Accepted | 0.585 | 3.217 | 0.475 | 0.007 | 0 | 0.27 | 0.06 | Rejected |
| 43 | 406.07 | Accepted | 0.718 | 3.056 | 0.223 | 0.003 | 0 | 0.867 | 0.044 | Accepted |
| 44 | 422.05 | Accepted | 0.645 | 3.205 | 0.207 | 0.002 | 0 | 0.858 | 0.059 | Rejected |
| 45 | 400.0 | Accepted | 0.709 | 2.977 | 0.364 | 0.003 | 0 | 0.13 | 0.04 | Accepted |
| 46 | 415.98 | Accepted | 0.642 | 3.139 | 0.401 | 0.005 | 0 | 0.113 | 0.035 | Rejected |
| 47 | 384.03 | Accepted | 0.724 | 3.012 | 0.349 | 0.007 | 0 | 0.172 | 0.039 | Accepted |
| 48 | 400.01 | Accepted | 0.666 | 3.192 | 0.387 | 0.007 | 0 | 0.154 | 0.023 | Accepted |
| 49 | 374.13 | Accepted | 0.747 | 3.064 | 0.237 | 0.006 | 0 | 0.666 | 0.029 | Accepted |
| 50 | 390.11 | Accepted | 0.694 | 3.231 | 0.217 | 0.006 | 0 | 0.741 | 0.045 | Rejected |
| 51 | 388.06 | Accepted | 0.729 | 3.003 | 0.41 | 0.011 | 0 | 0.413 | 0.125 | Accepted |
| 52 | 404.04 | Accepted | 0.657 | 3.192 | 0.448 | 0.018 | 0 | 0.253 | 0.134 | Rejected |
| 53 | 350.19 | Accepted | 0.785 | 3.027 | 0.22 | 0.004 | 0 | 0.872 | 0.122 | Accepted |
| 54 | 366.16 | Accepted | 0.711 | 3.2 | 0.195 | 0.002 | 0 | 0.832 | 0.18 | Rejected |
| 55 | 324.17 | Accepted | 0.78 | 2.909 | 0.487 | 0.009 | 0 | 0.377 | 0.048 | Accepted |
| 56 | 340.15 | Accepted | 0.725 | 3.097 | 0.541 | 0.005 | 0 | 0.235 | 0.069 | Rejected |
| 57 | 335.15 | Accepted | 0.763 | 3.078 | 0.605 | 0.007 | 0 | 0.771 | 0.037 | Accepted |
| 58 | 351.13 | Accepted | 0.714 | 3.259 | 0.643 | 0.004 | 0 | 0.539 | 0.042 | Accepted |
| 59 | 328.14 | Accepted | 0.777 | 2.969 | 0.355 | 0.005 | 0 | 0.735 | 0.05 | Accepted |
| 60 | 344.12 | Accepted | 0.723 | 3.158 | 0.344 | 0.007 | 0 | 0.504 | 0.077 | Rejected |
| 61 | 378.14 | Accepted | 0.742 | 3.032 | 0.194 | 0.005 | 0 | 0.651 | 0.068 | Accepted |
| 62 | 394.12 | Accepted | 0.663 | 3.203 | 0.179 | 0.004 | 0 | 0.539 | 0.058 | Rejected |
| 63 | 344.12 | Accepted | 0.772 | 2.963 | 0.346 | 0.003 | 0 | 0.285 | 0.057 | Accepted |
| 64 | 360.09 | Accepted | 0.705 | 3.152 | 0.362 | 0.002 | 0 | 0.144 | 0.054 | Rejected |
| 65 | 360.15 | Accepted | 0.777 | 3.142 | 0.258 | 0.011 | 0 | 0.054 | 0.047 | Accepted |
| 66 | 376.13 | Accepted | 0.7 | 3.317 | 0.239 | 0.011 | 0 | 0.069 | 0.06 | Rejected |
| 67 | 374.17 | Accepted | 0.763 | 3.163 | 0.213 | 0.009 | 0 | 0.46 | 0.082 | Accepted |
| 68 | 390.14 | Accepted | 0.679 | 3.334 | 0.205 | 0.008 | 0 | 0.289 | 0.062 | Rejected |
| 69 | 352.2 | Accepted | 0.784 | 2.991 | 0.301 | 0.05 | 0 | 0.418 | 0.102 | Accepted |
| 70 | 368.18 | Accepted | 0.702 | 3.166 | 0.292 | 0.036 | 0 | 0.308 | 0.132 | Rejected |
| 71 | 394.14 | Accepted | 0.735 | 3.072 | 0.299 | 0.027 | 0 | 0.919 | 0.099 | Accepted |
| 72 | 410.11 | Accepted | 0.659 | 3.236 | 0.281 | 0.018 | 0 | 0.863 | 0.076 | Rejected |
| 73 | 376.15 | Accepted | 0.752 | 3.07 | 0.311 | 0.022 | 0 | 0.577 | 0.061 | Accepted |
| 74 | 392.12 | Accepted | 0.683 | 3.239 | 0.316 | 0.018 | 0 | 0.441 | 0.062 | Rejected |
| 75 | 428.02 | Accepted | 0.678 | 2.971 | 0.127 | 0.007 | 0 | 0.84 | 0.143 | Accepted |
| 76 | 444.0 | Accepted | 0.598 | 3.147 | 0.121 | 0.008 | 0 | 0.863 | 0.07 | Rejected |
| 77 | 390.14 | Accepted | 0.744 | 2.996 | 0.06 | 0.004 | 0 | 0.946 | 0.192 | Accepted |
| 78 | 406.12 | Accepted | 0.655 | 3.158 | 0.053 | 0.001 | 0 | 0.951 | 0.065 | Rejected |
| 79 | 392.16 | Accepted | 0.74 | 2.97 | 0.089 | 0.014 | 0 | 0.854 | 0.1 | Accepted |
| 80 | 408.13 | Accepted | 0.641 | 3.134 | 0.08 | 0.013 | 0 | 0.839 | 0.058 | Rejected |
| 81 | 384.07 | Accepted | 0.735 | 2.923 | 0.121 | 0.002 | 0 | 0.703 | 0.056 | Accepted |
| 82 | 400.05 | Accepted | 0.655 | 3.098 | 0.12 | 0.002 | 0 | 0.743 | 0.03 | Rejected |
| 83 | 418.1 | Accepted | 0.647 | 2.995 | 0.059 | 0.003 | 0 | 0.878 | 0.211 | Accepted |
| 84 | 434.07 | Accepted | 0.475 | 3.155 | 0.052 | 0.003 | 0 | 0.931 | 0.059 | Rejected |
| 85 | 368.1 | Accepted | 0.702 | 2.935 | 0.107 | 0.003 | 0 | 0.831 | 0.077 | Accepted |
| 86 | 384.08 | Accepted | 0.54 | 3.111 | 0.097 | 0.003 | 0 | 0.889 | 0.041 | Rejected |
| 87 | 416.1 | Accepted | 0.658 | 3.048 | 0.108 | 0.009 | 0 | 0.84 | 0.231 | Accepted |
| 88 | 432.08 | Accepted | 0.495 | 3.206 | 0.103 | 0.007 | 0 | 0.915 | 0.061 | Rejected |
| 89 | 434.09 | Accepted | 0.638 | 3.054 | 0.111 | 0.011 | 0 | 0.943 | 0.415 | Accepted |
| 90 | 450.07 | Accepted | 0.472 | 3.209 | 0.105 | 0.011 | 0 | 0.964 | 0.116 | Rejected |
| 91 | 427.96 | Accepted | 0.676 | 3.016 | 0.256 | 0.002 | 0 | 0.605 | 0.192 | Accepted |
| 92 | 443.93 | Accepted | 0.586 | 3.186 | 0.278 | 0.002 | 0 | 0.476 | 0.226 | Rejected |
| 93 | 390.08 | Accepted | 0.741 | 3.016 | 0.131 | 0.001 | 0 | 0.927 | 0.246 | Accepted |
| 94 | 406.05 | Accepted | 0.64 | 3.171 | 0.107 | 0.001 | 0 | 0.912 | 0.331 | Rejected |
| 95 | 390.08 | Accepted | 0.74 | 3.206 | 0.478 | 0.013 | 0 | 0.295 | 0.18 | Accepted |
| 96 | 374.05 | Accepted | 0.742 | 3.046 | 0.464 | 0.008 | 0 | 0.35 | 0.119 | Accepted |
| 97 | 393.99 | Accepted | 0.721 | 2.862 | 0.238 | 0.003 | 0 | 0.685 | 0.139 | Accepted |
| 98 | 411.99 | Accepted | 0.699 | 3.041 | 0.266 | 0.005 | 0 | 0.464 | 0.154 | Accepted |
| 99 | 396.01 | Accepted | 0.717 | 3.016 | 0.277 | 0.028 | 0 | 0.472 | 0.107 | Accepted |
| 100 | 433.18 | Accepted | 0.681 | 3.019 | 0.053 | 0.005 | 0 | 0.965 | 0.871 | Accepted |
| 101 | 381.23 | Accepted | 0.703 | 3.281 | 0.208 | 0.04 | 0 | 0.943 | 0.929 | Accepted |
| 102 | 393.23 | Accepted | 0.731 | 3.036 | 0.152 | 0.004 | 0 | 0.848 | 0.62 | Accepted |
| 103 | 390.1 | Accepted | 0.736 | 3.082 | 0.228 | 0.004 | 0 | 0.745 | 0.038 | Accepted |
| 104 | 370.16 | Accepted | 0.754 | 3.074 | 0.274 | 0.011 | 0 | 0.671 | 0.04 | Accepted |
| 105 | 424.13 | Accepted | 0.65 | 3.187 | 0.15 | 0.005 | 0 | 0.912 | 0.055 | Accepted |
| 106 | 397.2 | Accepted | 0.725 | 3.042 | 0.139 | 0.002 | 0 | 0.862 | 0.672 | Accepted |
| 107 | 380.2 | Accepted | 0.749 | 3.681 | 0.283 | 0.028 | 0 | 0.555 | 0.036 | Accepted |
| 108 | 380.2 | Accepted | 0.69 | 3.022 | 0.215 | 0.093 | 0 | 0.371 | 0.167 | Accepted |
| 109 | 380.2 | Accepted | 0.688 | 2.965 | 0.147 | 0.013 | 0 | 0.32 | 0.021 | Accepted |
| 110 | 480.25 | Accepted | 0.514 | 3.245 | 0.105 | 0.916 | 0 | 0.139 | 0.238 | Accepted |
| 111 | 484.22 | Accepted | 0.509 | 3.25 | 0.088 | 0.818 | 0 | 0.237 | 0.257 | Accepted |

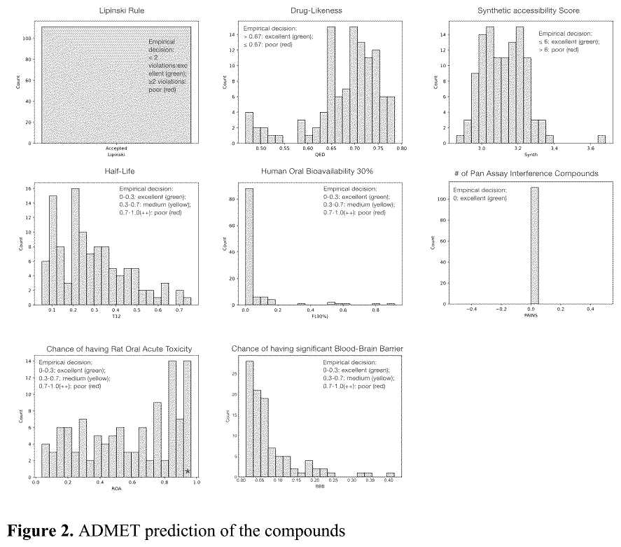

The Rat Oral Acute Toxicity plot in FIG. 2 has the predicted toxicity of MCC950, a well-studied NLRP3 inhibitor which is potent but failed in the clinical trials due to toxicity. The graph shows most compounds in Table 1 are expected to less toxic than MCC950.

FIG. 2. Statistical plots summarizing the ADMET prediction of the compounds enlisted in Table 1 by a machine learning model.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous to the compounds of this invention also can be made, screened for their efficacy in treating a condition that relates to diseases and disorders mediated by NLRP3. Thus, other embodiments are also within the claims.

What is claimed is:

1. A compound of formula I or a pharmaceutically acceptable salt, solvate, clathrate, hydrate, stereoisomer, or tautomer thereof. 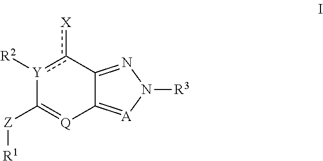
Wherein:
A is Nor CH;
Q is N or CH;
Z is O, S, N(R^{a}) or C(R^{b})(R^{c});
R^{a} is hydrogen, C₁-C₆ alkyl, C₁-C₆ haloalkyl, C₃-C₈ cycloalkyl, 3- to 8-membered heterocycloalkyl, C₆-C₁₀ aryl, or 5- to 9-membered heteroaryl;
or R^{a}, together with R¹ and the nitrogen atom to which it is attached, forms a 3- to 8-membered heterocycloalkyl;
R^{b} is hydrogen or C₁-C₆ alkyl;
R^{c} is hydrogen or C₁-C₆ alkyl; or R^{b} and R^{c}, together with the atoms to which they are attached, form a C₃-C₈ cycloalkyl;
R¹ is C₁-C₆ alkyl, C₂-C₆ alkenyl, C₂-C₆ alkynyl, C₁-C₆ haloalkyl, —(CH₂)ₙ—(C₃-C₁₀ cycloalkyl), —(CH₂)ₙ—(C₆-C₁₀ aryl), —(CH₂)ₙ-(3- to 8-membered heterocycloalkyl), or —(C₂)ₙ-(5- to 9-membered heteroaryl), —R⁴SR⁶, —R⁴C(O)OR⁶, —R⁴C(O)N(R⁷)(R⁸), —R⁴N(R⁷)C(O)R⁸, —R⁴S(O)ₜR⁵, —R⁴S(O)ₜN(R⁷)(R⁸), —R⁴N(R⁷)S(O)ₜ(R⁸), or —R⁴P(O)(R⁵)₂, wherein the C₁-C₆ alkyl, C₂-C₆ alkenyl, C₂-C₆ alkynyl, C₁-C₆ haloalkyl, C₃-C₈ cycloalkyl, 3- to 8-membered heterocycloalkyl, C₆-C₁₀ aryl, or 5- to 9-membered heteroaryl is optionally substituted with one more halo, CN, C₁-C₆ alkyl, —NR⁵R⁶, —R⁴OR⁵, an optionally substituted 3- to 8-membered heterocycloalkyl, an optionally substituted C₆-C₁₀ aryl, or an optionally substituted 5- to 9-membered heteroaryl;
R² is absent, hydrogen, deuterium, halo, C₁-C₆ alkyl or C₁-C₆ haloalkyl, —(CH₂)ₙ—(C₃-C₁₀ cycloalkyl), —(CH₂)ₙ—(C₆-C₁₀ aryl), —(CH₂)ₙ-(3- to 8-membered heterocycloalkyl), or —(CH₂)ₙ-(5- to 9-membered heteroaryl), —R⁴SR⁶, —R⁴C(O)OR⁶, —R⁴C(O)N(R⁷)(R⁸), —R⁴N(R⁷)C(O)R⁸, —R⁴S(O)ₜR⁵, —R⁴S(O)ₜN(R⁷)(R⁸), —R⁴N(R⁷)S(O)ₜ(R⁸), —R⁴OC(O)R⁵ or —R⁴P(O)(R⁵)₂, wherein the C₁-C₆ alkyl, C₂-C₆ alkenyl, C₂-C₆ alkynyl, C₁-C₆ haloalkyl, C₃-C₈ cycloalkyl, 3- to 8-membered heterocycloalkyl, C₆-C₁₀ aryl, or 5- to 9-membered heteroaryl is optionally substituted with one more halo, CN, C₁-C₆ alkyl, —NR⁵R⁶, —R⁴OR⁵, an optionally substituted 3- to 8-membered heterocycloalkyl, an optionally substituted C₆-C₁₀ aryl, or an optionally substituted 5- to 9-membered heteroaryl;
Y is N, O, S, or C(R^{b});
X is N(R^{a}), O, S, or C(R^{b})(R^{c});
R³ is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more C₁-C₆ alkyl, C₁-C₆ haloalkyl, C₃-C₈ cycloalkyl, 3- to 8-membered heterocycloalkyl, C₆-C₁₀ aryl, 5- to 9-membered heteroaryl, halo, cyano, —R⁴OR⁵, —R⁴N(R⁵)(R⁶), —R⁴SR⁶, —R⁴C(O)OR⁶, —R⁴C(O)N(R⁷)(R⁸), —R⁴N(R⁷)C(O)R⁸, —R⁴S(O)ₜR⁵, —R⁴S(O)ₜN(R⁷)(R⁸), R⁴N(R⁷)S(O)ₜ(R⁸), —R⁴P(O)(R⁵)₂, or —R⁴SF₅;
R⁴ is a bond, C₁-C₆ alkyl, C₂-C₆ alkenyl, or C₂-C₆ alkynyl;
R⁵ is hydrogen, C₁-C₆ alkyl, C₁-C₆ haloalkyl, C₃-C₈ cycloalkyl, 3- to 8-membered heterocycloalkyl, C₆-C₁₀ aryl, or 5- to 9-membered heteroaryl, wherein the alkyl is optionally substituted with one or more D;
R⁶ is hydrogen, C₁-C₆ alkyl, C₁-C₆ haloalkyl, C₃-C₈ cycloalkyl, 3- to 8-membered heterocycloalkyl, C₆-C₁₀ aryl, or 5- to 9-membered heteroaryl;
R⁷ is hydrogen, C₁-C₆ alkyl, C₁-C₆ haloalkyl, C₃-C₈ cycloalkyl, 3- to 8-membered heterocycloalkyl, C₆-C₁₀ aryl, or 5- to 9-membered heteroaryl;
R⁸ is hydrogen, C₁-C₆ alkyl, C₁-C₆ haloalkyl, C₃-C₈ cycloalkyl, 3- to 8-membered heterocycloalkyl, C₆-C₁₀ aryl, or 5- to 9-membered heteroaryl; or
R⁷ and R⁸ together with the atoms to which they are attached, form a 3- to 8-membered heterocycloalkyl;
n is 0, 1, 2, 3, or 4;
and t is 1 or 2.

2. The compound according to claim 1, represented by formula II, or a pharmaceutically acceptable salt, solvate, clathrate, hydrate, stereoisomer, or tautomer thereof. 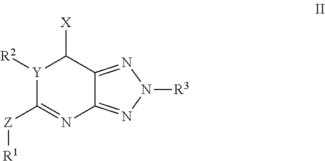
Wherein:
Z is O, S, N(R^{a}) or C(R^{b})(R^{c});
R^{a} is hydrogen, C₁-C₆ alkyl, C₁-C₆ haloalkyl, C₃-C₈ cycloalkyl, 3- to 8-membered heterocycloalkyl, C₆-C₁₀ aryl, or 5- to 9-membered heteroaryl;
or R^{a}, together with R¹ and the nitrogen atom to which it is attached, forms a 3- to 8-membered heterocycloalkyl;
R^{b} is hydrogen or C₁-C₆ alkyl;
R^{c} is hydrogen or C₁-C₆ alkyl; or R^{b} and R^{c}, together with the atoms to which they are attached, form a C₃-C₈ cycloalkyl;
R¹ is C₁-C₆ alkyl, C₂-C₆ alkenyl, C₂-C₆ alkynyl, C₁-C₆ haloalkyl, —(CH₂)ₙ—(C₃-C₁₀ cycloalkyl), —(CH₂)ₙ—(C₆-C₁₀ aryl), —(CH₂)ₙ-(3- to 8-membered heterocycloalkyl), or —(CH₂)ₙ-(5- to 9-membered heteroaryl), —R⁴SR⁶, —R⁴C(O)OR⁶, —R⁴C(O)N(R⁷)(R⁸), —R⁴N(R⁷)C(O)R⁸, —R⁴S(O)ₜR⁵, —R⁴S(O)ₜN(R⁷)(R⁸), —R⁴N(R⁷)S(O)ₜ(R⁸), or —R⁴P(O)(R⁵), wherein the C₁-C₆ alkyl, C₂-C₆ alkenyl, C₂-C₆ alkynyl, C₁-C₆ haloalkyl, C₃-C₈ cycloalkyl, 3- to 8-membered heterocycloalkyl, C₆-C₁₀ aryl, or 5- to 9-membered heteroaryl is optionally substituted with one more halo, CN, C₁-C₆ alkyl, —NR⁵R⁶, —R⁴OR⁵, an optionally substituted 3- to 8-membered heterocycloalkyl, an optionally substituted C₆-C₁₀ aryl, or an optionally substituted 5- to 9-membered heteroaryl;
R² is absent, hydrogen, deuterium, halo, C₁-C₆ alkyl or C₁-C₆ haloalkyl, —(CH₂)ₙ—(C₃-C₁₀ cycloalkyl), —(CH₂)ₙ—(C₆-C₁₀ aryl), —(CH₂)ₙ-(3- to 8-membered heterocycloalkyl), or —(CH₂)ₙ-(5- to 9-membered heteroaryl), —R⁴SR⁶, —R⁴C(O)OR⁶, —R⁴C(O)N(R⁷)(R⁸), —R⁴N(R⁷)C(O)R⁸, —R⁴S(O)ₜR⁵, —R⁴S(O)ₜN(R⁷)(R⁸), —R⁴N(R⁷)S(O)ₜ(R⁸), —R⁴OC(O)R⁵ or —R⁴P(O)(R⁵)₂, wherein the C₁-C₆ alkyl, C₂-C₆ alkenyl, C₂-C₆ alkynyl, C₁-C₆ haloalkyl, C₃-C₈ cycloalkyl, 3- to 8-membered heterocycloalkyl, C₆-C₁₀ aryl, or 5- to 9-membered heteroaryl is optionally substituted with one more halo, CN, C₁-C₆ alkyl, —NR⁵R⁶, —R⁴OR⁵, an optionally substituted 3- to 8-membered heterocycloalkyl, an optionally substituted C₆-C₁₀ aryl, or an optionally substituted 5- to 9-membered heteroaryl;
X is N(R^{a}), O, S, or C(R^{b})(R^{c});
R³ is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more C₁-C₆ alkyl, C₁-C₆ haloalkyl, C₃-C₈ cycloalkyl, 3- to 8-membered heterocycloalkyl, C₆-C₁₀ aryl, 5- to 9-membered heteroaryl, halo, cyano, —R⁴OR⁵, —R⁴N(R⁵)(R⁶), —R⁴SR⁶, —R⁴C(O)OR⁶, —R⁴C(O)N(R⁷)(R⁸), —R⁴N(R⁷)C(O)R⁸, —R⁴S(O)ₜR⁵, —R⁴S(O)ₜN(R⁷)(R⁸), R⁴N(R⁷)S(O)ₜ(R⁸), —R⁴P(O)(R⁵)₂, or —R⁴SF₅;
R⁴ is a bond, C₁-C₆ alkyl, C₂-C₆ alkenyl, or C₂-C₆ alkynyl;
R⁵ is hydrogen, C₁-C₆ alkyl, C₁-C₆ haloalkyl, C₃-C₈ cycloalkyl, 3- to 8-membered heterocycloalkyl, C₆-C₁₀ aryl, or 5- to 9-membered heteroaryl, wherein the alkyl is optionally substituted with one or more D;
R⁶ is hydrogen, C₁-C₆ alkyl, C₁-C₆ haloalkyl, C₃-C₈ cycloalkyl, 3- to 8-membered heterocycloalkyl, C₆-C₁₀ aryl, or 5- to 9-membered heteroaryl;
R⁷ is hydrogen, C₁-C₆ alkyl, C₁-C₆ haloalkyl, C₃-C₈ cycloalkyl, 3- to 8-membered heterocycloalkyl, C₆-C₁₀ aryl, or 5- to 9-membered heteroaryl;
R⁸ is hydrogen, C₁-C₆ alkyl, C₁-C₆ haloalkyl, C₃-C₈ cycloalkyl, 3- to 8-membered heterocycloalkyl, C₆-C₁₀ aryl, or 5- to 9-membered heteroaryl; or
R⁷ and R⁸ together with the atoms to which they are attached, form a 3- to 8-membered heterocycloalkyl;
n is 0, 1, 2, 3, or 4;
and t is 1 or 2.

3. The compound according to any one of claims 1 to 2, represented by formula III, or a pharmaceutically acceptable salt, solvate, clathrate, hydrate, stereoisomer, or tautomer thereof. 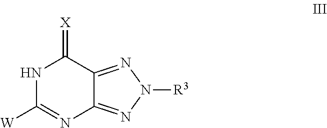
Wherein:
X is N(R^{a}), O, S, or C(R^{b})(R^{c});
R³ is C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more C₁-C₆ alkyl, C₁-C₆ haloalkyl, C₃-C₈ cycloalkyl, 3- to 8-membered heterocycloalkyl, C₆-C₁₀ aryl, 5- to 9-membered heteroaryl, halo, cyano, —R⁴OR⁵, —R⁴N(R⁵)(R⁶), —R⁴SR⁶, —R⁴C(O)OR⁶, —R⁴C(O)N(R⁷)(R⁸), —R⁴N(R⁷)C(O)R⁸, —R⁴S(O)ₜR⁵, —R⁴S(O)ₜN(R⁷)(R⁸), R⁴N(R⁷)S(O)ₜ(R⁸), —R⁴P(O)(R⁵)₂, or —R⁴SF₅;
W is hydrogen, amino, C₁-C₆ alkyl, C₁-C₆ haloalkyl, C₃-C₈ cycloalkyl, 3- to 8-membered heterocycloalkyl, C₆-C₁₀ aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more C₁-C₆ alkyl, C₁-C₆ haloalkyl, C₃-C₈ cycloalkyl, 3- to 8-membered heterocycloalkyl, C₆-C₁₀ aryl, 5- to 9-membered heteroaryl, halo, cyano, —R⁴OR⁵, —R⁴N(R⁵)(R⁶), —R⁴SR⁶, —R⁴C(O)OR⁶, —R⁴C(O)N(R⁷)(R⁸), —R⁴N(R⁷)C(O)R⁸, —R⁴S(O)ₜR⁵, —R⁴S(O)ₜN(R⁷)(R⁸), R⁴N(R⁷)S(O)ₜ(R⁸), —R⁴P(O)(R⁵)₂, or —R⁴SF₅;
R⁴ is a bond, C₁-C₆ alkyl, C₂-C₆ alkenyl, or C₂-C₆ alkynyl;
R⁵ is hydrogen, C₁-C₆ alkyl, C₁-C₆ haloalkyl, C₃-C₈ cycloalkyl, 3- to 8-membered heterocycloalkyl, C₆-C₁₀ aryl, or 5- to 9-membered heteroaryl, wherein the alkyl is optionally substituted with one or more D;
R⁶ is hydrogen, C₁-C₆ alkyl, C₁-C₆ haloalkyl, C₃-C₈ cycloalkyl, 3- to 8-membered heterocycloalkyl, C₆-C₁₀ aryl, or 5- to 9-membered heteroaryl;
R⁷ is hydrogen, C₁-C₆ alkyl, C₁-C₆ haloalkyl, C₃-C₈ cycloalkyl, 3- to 8-membered heterocycloalkyl, C₆-C₁₀ aryl, or 5- to 9-membered heteroaryl;
R⁸ is hydrogen, C₁-C₆ alkyl, C₁-C₆ haloalkyl, C₃-C₈ cycloalkyl, 3- to 8-membered heterocycloalkyl, C₆-C₁₀ aryl, or 5- to 9-membered heteroaryl; or
R⁷ and R⁸ together with the atoms to which they are attached, form a 3- to 8-membered heterocycloalkyl;
n is 0, 1, 2, 3, or 4;
and t is 1 or 2.

4. A pharmaceutical composition comprising the compound or a pharmaceutically acceptable salt, solvate, clathrate, hydrate, stereoisomer, or tautomer thereof according to any one of claims 1 to 3 and a pharmaceutically acceptable carrier.

5. An NLRP3 inflammasome inhibitor comprising the compound according to any one of claims 1 to 3 or a pharmaceutically acceptable salt, solvate, clathrate, hydrate, stereoisomer, or tautomer thereof.

6. Multiple sclerosis, inflammatory bowel disease, arteriosclerosis, cryopyrin-associated periodic fever syndrome, non-alcoholic steatohepatitis, comprising a compound according to any one of claims 1 to 3 or a pharmaceutically acceptable salt, solvate, clathrate, hydrate, stereoisomer, or tautomer thereof, gout, ischemic heart disease, Alzheimer's disease, Parkinson's disease, amyotrophic lateral sclerosis and traumatic brain injury.

7. The therapeutic or preventive agent according to claim 6, wherein the inflammatory bowel disease is ulcerative colitis or Crohn's disease.

8. The therapeutic or prophylactic agent according to claim 6, wherein the cryopyrin-associated periodic fever syndrome is familial cold autoinflammatory syndrome, Muckle-Wells syndrome, chronic infantile neurocutaneous joint syndrome, or neonatal-onset multisystem inflammatory disease.

9. A method of inhibiting the NLRP3 inflammasome, comprising administering a therapeutically effective amount of the compound according to any one of claim 1 to 3 or a pharmaceutically acceptable salt, solvate, clathrate, hydrate, stereoisomer, or tautomer thereof to a mammal.

10. Multiple sclerosis, inflammatory bowel disease, arteriosclerosis, comprising administering a therapeutically effective amount of a compound according to any one of claims 1 to 3 or a pharmaceutically acceptable salt, solvate, clathrate, hydrate, stereoisomer, or tautomer thereof to a mammal, cryopyrin-associated periodic fever syndrome, non-alcoholic steatohepatitis, gout, ischemic heart disease, Alzheimer's disease, Parkinson's disease, amyotrophic lateral sclerosis and traumatic brain injury.

11. The method according to claim 10, wherein the inflammatory bowel disease is ulcerative colitis or Crohn's disease.

12. The method according to claim 10, wherein the cryopyrin-associated periodic fever syndrome is familial cold autoinflammatory syndrome, Muckle-Wells syndrome, chronic infantile neurocutaneous joint syndrome, or neonatal-onset multisystem inflammatory disease.

13. Use of the compound according to any one of claims 1 to 3 or a pharmaceutically acceptable salt, solvate, clathrate, hydrate, stereoisomer, or tautomer thereof for manufacturing an NLRP3 inflammasome inhibitor.

14. Multiple sclerosis, inflammatory bowel disease, arteriosclerosis, cryopyrin-associated periodic fever syndrome, non-alcoholic steatohepatitis, gout, ischemic heart disease, Alzheimer's disease, Parkinson's disease, amyotrophic lateral sclerosis and brain trauma 13. Use of the compound according to any one of claims 1 to 3 or a pharmaceutically acceptable salt, solvate, clathrate, hydrate, stereoisomer, or tautomer thereof for manufacturing a therapeutic or prophylactic agent for a disease selected from the group consisting of injuries.

15. The use according to claim 14, wherein the inflammatory bowel disease is ulcerative colitis or Crohn's disease.

16. The use according to claim 14, wherein the cryopyrin-associated periodic fever syndrome is familial cold autoinflammatory syndrome, Muckle-Wells syndrome, chronic infantile neurocutaneous joint syndrome or neonatal-onset multisystem inflammatory disease.

17. A compound according to any one of claims 1 to 3 or a pharmaceutically acceptable salt, solvate, clathrate, hydrate, stereoisomer, or tautomer thereof for use in inhibiting the NLRP3 inflammasome.

18. Multiple sclerosis, inflammatory bowel disease, arteriosclerosis, cryopyrin-associated periodic fever syndrome, non-alcoholic steatohepatitis, gout, ischemic heart disease, Alzheimer's disease, Parkinson's disease, amyotrophic lateral sclerosis and brain trauma 13. A compound according to any one of claims 1 to 3, or a pharmaceutically acceptable salt, solvate, clathrate, hydrate, stereoisomer, or tautomer thereof, for use in treating or preventing a disease selected from the group consisting of injuries.

19. The compound or a pharmaceutically acceptable salt, solvate, clathrate, hydrate, stereoisomer, or tautomer thereof according to claim 18, wherein the inflammatory bowel disease is ulcerative colitis or Crohn's disease.

20. The compound of claim 18, wherein the cryopyrin-associated periodic fever syndrome is Familial Cold Autoinflammatory Syndrome, Muckle Wells Syndrome, Chronic Infantile Neurocutaneous Joint Syndrome, or Neonatal Onset Multisystem Inflammatory Disease, or a pharmaceutically acceptable salt, solvate, clathrate, hydrate, stereoisomer, or tautomer thereof to be served.
